(19) United States
(12) Patent Application Publication
(10) Pub. No.: US 2026/0256808 A1
(43) Pub. Date: Sep. 3, 2026

(54) Istaroxime-Containing Intravenous Formulation for the Treatment of Pre-Cardiogenic Shock and Cardiogenic Shock

(71) Applicant: Windtree Therapeutics, Inc., Warrington, PA (US)

(72) Inventor: Steven SIMONSON, Kennett Square, PA (US)

(73) Assignee: Windtree Therapeutics, Inc., Warrington, PA (US)

(21) Appl. No.: 18/858,086

(22) PCT Filed: Apr. 18, 2023
(86) PCT No.: PCT/US23/18999

§ 371 Date: Oct. 18, 2024

Related U.S. Application Data
(60) Provisional application No. 63/332,909, filed on Apr. 20, 2022.

(51) Int. Cl. A61K 31/5685; A61K 9/00; A61K 31/138; A61K 31/197; A61K 31/216; A61K 31/277; A61K 31/341; A61K 31/403; A61K 31/41; A61K 31/4184; A61K 31/44; A61K 31/4422; A61P 9/00

(52) U.S. Cl. CPC A61K 31/5685; A61K 9/0019; A61K 31/138; A61K 31/197; A61K 31/216; A61K 31/277; A61K 31/341; A61K 31/403; A61K 31/41; A61K 31/4184; A61K 31/44; A61K 31/4422; A61P 9/00

(57) ABSTRACT

Compositions for intravenous infusion of istaroxime in human patients suffering from pre-cardiogenic shock or cardiogenic shock are disclosed. Likewise, methods of administering istaroxime to individuals exhibiting symptoms of, or having been diagnosed with pre-cardiogenic shock or cardiogenic shock are disclosed. In particular, disclosed herein is a method of treating or preventing pre-cardiogenic shock or cardiogenic shock in an individual by administering to the individual a dosage regimen of istaroxime by intravenous infusion for up to 24 hours.

### CROSS-REFERENCE TO RELATED APPLICATIONS

This claims the benefit of the filing date of U.S. Provisional Application No. 63/332,909, filed Apr. 20, 2022, the entire content of which is incorporated by reference herein.

### FILED OF THE INVENTION

The present invention relates to the field of pharmaceuticals, in particular to an istaroxime-containing intravenous formation for use in the treatment of pre-cardiogenic shock or cardiogenic shock.

### BACKGROUND

The prevalence of heart failure (HF) is age-dependent, ranging from less than 2% of people younger than 60 years of age to more than 10% of individuals older than 75 years of age [Metra & Teerlink, 2017, Lancet 390:1981-1995]. Most patients with a HF have a history of hypertension, coronary artery disease, cardiomyopathies, valve disease, or a combination of these disorders [Metra & Teerlink, 2017, Lancet 390:1981-1995]. Chronic heart failure (CHF) can be distinguished from individuals exhibiting acute heart failure (AHF), which refers generally to a rapid onset or worsening of the symptoms and/or signs of HF requiring immediate treatment and/or hospitalization. Individuals with CHF or other pre-existing cardiomyopathy, may experience a sudden onset of worsening of their condition leading to specific type of AHF referred to as acute decompensated heart failure (ADHF) [Joseph et al., 2009, Tex. Heart Inst. J. 36:510-520]. Importantly, the clinical course and prognosis outcomes for individuals with CHF is much worse after an episode of AHF or ADHF [Solomon et al., 2007, Circulation 116:1482-1487; Teneggi et al., 2018, Heart Failure Rev. 23:667-691; Joseph et al., 2009, Tex. Heart Inst. J. 36:510-520].

One of the most serious acute coronary conditions is cardiogenic shock, which is associated with high rates of morbidity and mortality, posing a therapeutic challenge for clinicians [van Diepen et al., 2017, Circulation 136: e232-e268; Hunziker et al., Circ. Cardiovasc. Interv. 12 (4): e007293; Berg et al., 2019, Circ. Outcomes 12: e005618]. Given that short-term mortality ranges from 35% to over 60% [Singh et al., 2019, Cardio. Rev. 27 (4): 198-201; Jentzer et al., 2019, JACC 74 (17): 2117-2127], interventions to prevent deterioration to overt cardiogenic shock are clearly needed. Other than revascularization of culprit coronary vessels in patients presenting with acute myocardial infarction (AMI), no other intervention has impacted short-term survival in patients with cardiogenic shock, and no established therapies exist for patients with non-AMI cardiogenic etiologies [Singh et al., 2019, Cardio. Rev. 27 (4): 198-201]. However, earlier intervention that could mitigate clinical and hemodynamic deterioration to cardiogenic shock may offer a window of opportunity to stabilize and reverse myocardial dysfunction as well as maintain favorable hemodynamics.

The period of clinical and hemodynamic deterioration that precedes cardiogenic shock is known as pre-cardiogenic shock, or, according to the Society for Cardiovascular Angiography and Intervention (SCAI) classification system, Stage B cardiogenic shock [see Jentzer et al., 2019, JACC 74 (17): 2117-2127]. The major aim of an intervention in pre-cardiogenic shock is to interrupt or reverse the hemodynamic downward spiral that ultimately leads to cardiogenic shock and death. Left unattended, pre-cardiogenic shock leads to overt cardiogenic shock with refractory hypotension, profoundly altered cellular metabolism, and end-organ failure. This transition period lacks the clinical and hemodynamic criteria that define cardiogenic shock, or SCAI Stage C cardiogenic shock; namely, systolic blood pressure (SBP) below 90 mmHg (or vasopressor therapy to maintain SBP over 90 mmHg), cardiac index (CI) less than 2.2 L/min/m², pulmonary capillary wedge pressure (PCWP) over 15 mmHg, and clinical evidence of end-organ hypoperfusion [Hochman et al., 1999, N. Engl. J. Med. 341 (9): 625-634].

While AMI remains the most common cause of pre-cardiogenic shock, steady hemodynamic deterioration leading to ADHF with reduced ejection fraction (HFrEF) is the most prevalent cause of pre-cardiogenic shock in non-AMI patients [Singh et al., 2019, Cardio. Rev. 27 (4): 198-201; Savarese & Lund, 2017, Cardiac Failure Rev. 3 (1): 7-11]. Indeed, ADHF may account for up to 30% of all cardiogenic shock cases [Kar et al., 2011, J. Am Coll. Cardiol. 57 (6) 688-696]. These individuals often experience a decline in disease stability or often have poor adherence to guideline-based therapies, triggering an acute worsening of their condition. Treatment of patients with ADHF and cardiogenic shock or CHF presenting in cardiogenic shock can differ substantially from the treatment of other types of cardiogenic shock because the hemodynamic condition and neurohormonal milieu are often strikingly different. Patients with HF often have profound upregulation of vasoconstrictor substances, such as angiotensin II, endothelin 1, and norepinephrine [Milo-Cotter et al., 2011, Cardiology 119:96-105; Shah et al., 2001, Rev. Cardiovasc. Med. 2 (supp. 2) S2-S6].

Regardless of the underlying etiology, it has been reported that unloading of the left ventricular (LV) filling pressure significantly reduces LV end-diastolic wall stress and decompresses the microvasculature, thereby increasing the myocardial blood flow to the endocardial layers and preserving LV contractile function [Kapur et al., 2013, Circulation 128 (4): 328-336]. Therefore, pharmacological intervention that can stabilize the failing LV and improve myocardial contractility and hemodynamics without increasing workload or myocardial oxygen demand are most desirable to prevent the deterioration of pre-cardiogenic shock to overt cardiogenic shock. Currently, most pharmacologic interventions, which include inotropic agents (e.g., dobutamine, milrinone) and vasoactive agents (e.g., norepinephrine), as well as newer inotropic agents under development, have not shown to reduced mortality despite transient improvements in hemodynamic status. Furthermore, the treatment of cardiogenic shock is complicated by the heterogeneity of the underlying pathophysiological mechanisms causing the condition, and present medical therapies and interventions are unable to correctly account for individual patient variability in order to properly tailor pre-cardiogenic and cardiogenic shock treatment. Thus, there is an unmet need for an agent to treat individuals with pre-cardiogenic shock to prevent the hemodynamic deterioration that leads to overt cardiogenic shock without increasing mortality.

One compound currently being studied for the treatment of patients with AHF is Istaroxime (PST 2744), which is an androstenedione derivative and chemically unrelated to cardiac glycosides. Istaroxime may have potential to overcome may of the issues with prior or existing therapies being used to treat patients with AHF. Istaroxime exerts its effects through dual mechanisms of action: 1) the inhibition of the Na⁺/K⁺-ATPase activity, thereby causing an increase in intracellular calcium, which increases cardiomyocyte contractility (inotropy); and 2) the activation of the sarcoplasmic reticulum calcium ATPase isoform (SERCA2a), thus improving both myocardial relaxation (lusitropy) and contractility as well as potentially reducing the risk for arrythmias.

One particular clinical study that examined the effects of Istaroxime in patients with AHF is outlined in the HORIZON-HF Study [see Gheorghiade et al., JACC, 51 (23): 2276-2285]. This study looked at 120 patients hospitalized with ADHF and reduced LV systolic functions administered escalating doses of Istaroxime for 6 hours (i.e., 0.5-1.0-1.5 μg/kg/min). The primary endpoint of the study was the change from baseline in PCWP after 6 hours and revealed dose-dependent improvements in PCWP between the treatment cohort compared to the placebo group. However, this improvement in PCWP plateaued after 3 hours and remained constant from 3 hours to 6 hours following the initiation of the infusion. The results also demonstrated that HR tended to decrease during the 6-hour infusion, while SBP slightly increased in a dose-dependent manner. However, this study did not include ADHF patients presenting with persistent hypotension (the average baseline SBP was 116 mmHg and specifically excluded patients with an SBP lower than 90) or cardiogenic shock or pre-clinical shock, nor did it show the efficacy of Istaroxime to increase SBP to the extent necessary to alleviate the hypotension associated with ADHF patients with pre-cardiogenic shock.

WO 2020/180356 A9 discloses another clinical study that examined prolonged istaroxime infusion in 120 patients with ADHF. This study involved two doses of istaroxime (0.5 μg/kg/min and 1.0 μg/kg/min) infused for up to 24 hours. The primary endpoint of this study was E/Ea ratio and revealed an improvement in both treatment groups compared to the placebo groups. In contrast to what the results of the Horizon Study suggested, prolonged infusion with Istaroxime showed significant improvements for most of the diastolic function parameters measured after 6 hours of infusion. However, this study included only ADHF patients with SBP ranges between 90 mmHg and 120 mmHg thus excluding patients exhibiting pre-cardiogenic shock or cardiogenic shock.

Thus, there is a clear but unmet need to treat patients with pre-cardiogenic shock to stabilize the failing LV and prevent the hemodynamic and subsequently general metabolic deterioration that leads to overt cardiogenic shock.

### SUMMARY OF THE INVENTION

Described herein are pharmaceutical compositions containing istaroxime formulated for administration to a subject for use in a treatment method for pre-cardiogenic shock or cardiogenic shock. In particular embodiments, the administration is by intravenous infusion and is for a duration of up to about 24 hours, whereby the systolic blood pressure (SBP), diastolic blood pressure (DBP), mean blood pressure, and other parameters of heart function in the individual are improved. For instance, the improvement in heart function can be measured by a change from baseline of SBP or SBP area under the curve (AUC) at 6 hours and/or 24 hours from the start of infusion. Individuals with pre-cardiogenic shock or cardiogenic shock may exhibit improved heart function following administration of istaroxime (e.g., by intravenous infusion) for a period of up to 24 hours. Surprisingly, it has been discovered that individuals with pre-cardiogenic shock that were administered istaroxime by intravenous infusion exhibited a concomitant increase in both cardiac index and blood pressure (e.g., systolic blood pressure), which is unique and has not been observed with any previous intravenous drugs administered to individuals with pre-cardiogenic shock or cardiogenic shock.

One aspect of the invention features a pharmaceutical composition for use as a medicament in the treatment or prevention of early cardiogenic shock or cardiogenic shock in an individual. The individual is defined as having acute heart failure with a systolic blood pressure of less than about 90 mmHg for a first period of time of at least about 1 hour. The pharmaceutical composition includes a pharmaceutically acceptable carrier and istaroxime or a pharmaceutically acceptable salt, solvate, or hydrate thereof. The pharmaceutical composition is formulated for administration by intravenous infusion at a dose of about 0.2 μg/kg/min to about 2.0 μg/kg/min for a second period of time of at least about 3 hours.

In one embodiment, one or more parameters of heart function in the individual are measured, such as blood pressure. In another embodiment, the blood pressure is systolic blood pressure, and the administration of the pharmaceutical composition results in an increase over baseline for the systolic blood pressure. In other embodiments, the one or more parameters of heart function in the individual additionally includes cardiac index, left atrial area, stroke volume, left ventricular end-systolic volume, left ventricular end-diastolic volume, or any combination thereof. In some embodiments, the acute heart failure is acute decompensated heart failure without evidence of end-organ hypoperfusion or acute coronary syndrome. In other embodiments, the individual has a heart rate of about 75 beats/minute and about 150 beats/minute or about 60 bpm to about 150 bpm if the individual has been administered a beta blocker.

In one embodiments, the istaroxime or pharmaceutically acceptable salt, solvate, or hydrate thereof is formulated for administration by intravenous infusion at a dose of about 1.0 μg/kg/min to about 1.5 g/kg/min. In another embodiment, the first period of time is at least about 2 hours, or at least about 6 hours, or at least about 24 hours. In one embodiment, the individual has at least one of dyspnea at rest or minimal exertion, congestion on chest x-ray or lung ultrasound with brain natriuretic peptide of less than about 400 μg/mL or N-terminal pro b-type Natriuretic Peptide of at least 1,400 μg/ml, or left ventricular ejection fraction of less than about 40%, or echocardiogram confirming ejection fraction of less than about 40%. In yet another embodiment, the one or more parameters of heart function further comprise heart rate (HR), diastolic relaxation, systolic contraction, dyspnea, chest congestion, diastolic blood pressure (DBP), systolic blood pressure (SBP), creatine clearance, deceleration slope, mitral inflow velocity, mean arterial pressure, brain natriuretic peptide levels (BNP), NT-pro-BNP levels, troponin levels, venous lactate levels, echocardiographic measurements, left ventricle end diastolic diameter (EDD), left ventricle end systolic diameter (ESD), left ventricle end diastolic volume (EDV), left ventricle end systolic volume (ESV), left atrium diameter (LAD), left atrium area (LAA), left atrium volume (LAV), E wave, A wave, E wave deceleration time (EDT), E/A ratio, Ea, Aa, E/Ea ratio, left ventricle ejection fraction (LVEF), Sa, stroke volume (SV), cardiac output (CO), stroke volume index (SVI), cardiac index (CI), pulmonary arterial systolic pressure (PASP), tricuspid annular plane systolic excursion (TAPSE), right ventricle Sa, mitral regurgitation (MR), inferior vena cava diameter (IVC), or any combination thereof.

In other embodiments, the pharmaceutical composition includes one or more further therapeutically active ingredients, such as, but not limited to ACE inhibitors (e.g., Lisinopril or Ramipril), angiotensin receptor blockers (ARBs) (e.g., valsartan, candesartan, olmesartan, telmisartan, or losartan), diuretics (e.g., furosemide, bumetanide, torasemide, metolazone, an aldosterone antagonist, or a thiazide), Ca channel blockers, beta-blockers (e.g., carvedilol or metoprolol), digitalis, NO donors, vasodilators (e.g., hydralazine, amlodipine, felodipine, diltiazem, and verapamil), SERCA2a stimulators, neprilysin (NEP) inhibitors, myosin filament activators, recombinant relaxin-2 mediators, recombinant NP protein, activators of the soluble Guanylate Cyclase (sGC), and/or beta-arrestin ligand of Angiotensin II receptor (e.g., sacubitril). In another embodiment, the pharmaceutical composition comprises saline as the pharmaceutically acceptable carrier, and the istaroxime is reconstituted from a lyophilized powder comprising the istaroxime admixed with a bulking agent.

In another aspect of the invention, provided herein is a pharmaceutical composition for use as a medicament in the treatment of pre-cardiogenic shock or cardiogenic shock in an individual. The pharmaceutical composition includes a pharmaceutically acceptable carrier and istaroxime or a pharmaceutically acceptable salt, solvate, or hydrate thereof. The istaroxime or pharmaceutically acceptable salt, solvate, or hydrate thereof is formulated for administration by intravenous infusion at a dose of about 0.2 μg/kg/min to about 2.0 μg/kg/min for at least about 6 hour, whereby blood pressure and cardiac output are improved. In one embodiment, the istaroxime or pharmaceutically acceptable salt, solvate, or hydrate thereof is formulated for administration by intravenous infusion at a dose of about 1.0 μg/kg/min to about 1.5 μg/kg/min for at least about 24 hours.

In another embodiments, the blood pressure is systolic blood pressure, which can be measured by sphygmomanometer or arterial line. In one embodiment, the administration of the pharmaceutical composition increasing systolic blood pressure over baseline by 6 hours after start of intravenous infusion. In some embodiments, the administration of the pharmaceutical composition to the individual results in (a) increased cardia index; (b) decreased left atrial area; (c) increased stroke volume; (d) decreased left ventricular end-diastolic volume; (e) decreased left ventricular end-diastolic volume; or (f) any combination of (a)-(e). In an embodiment, the pharmaceutical composition includes saline as the pharmaceutically acceptable carrier, and the istaroxime is reconstituted from a lyophilized powder comprising the istaroxime admixed with a bulking agent. In another embodiment, the individual is undergoing a therapeutic treatment with one or more therapeutically active ingredients, such as, but not limited to ACE inhibitors, ARBs, diuretics, Ca channel blockers, beta-blockers, digitalis, NO donors, vasodilators, SERCA2a stimulators, neprilysin (NEP) inhibitors, myosin filament activators, recombinant relaxin-2 mediators, recombinant NP protein, activators of the soluble Guanylate Cyclase (sGC), and/or beta-arrestin ligand of Angiotensin II receptor.

Another aspect of the invention features a method of treating or preventing early cardiogenic shock or cardiogenic shock in an individual. The method includes the steps of providing an individual with acute heart failure, determining that the individual has a systolic blood pressure of less than about 90 mmHg for a first period of time of at least about 1 hour, and administering to the individual a therapeutically effective amount of a pharmaceutical composition. The pharmaceutical composition includes a pharmaceutically acceptable carrier and istaroxime or a pharmaceutically acceptable salt, solvate, or hydrate thereof, which is administered at a dose of about 0.2 μg/kg/min to about 2.0 μg/kg/min by infusion for a second period of time of at least about 3 hours. In such methods, the administering of the pharmaceutical composition results in an increase over baseline for both systolic heart function and cardiac output, the increase measurable by a third period of time following the start of the intravenous infusion, thereby treating or preventing early cardiogenic shock or cardiogenic shock in the individual.

In an embodiment, the method includes the step of measuring one or more parameters of heart function in the individual, such as blood pressure or, preferably, systolic blood pressure. In one embodiment, the administering of the pharmaceutical composition results in an increase over baseline for the systolic blood pressure, and the third period of time is about 6 hours. In another embodiment, the blood pressure is measured as systolic blood pressure area under the curve, and administering of the pharmaceutical composition results in an increase over baseline for the systolic blood pressure area under the curve—the third period of time is about 6 hours. In yet another embodiment, the one or more parameters of heart function in the individual additionally includes cardiac index, left atrial area, stroke volume, left ventricular end-systolic volume, left ventricular end-diastolic volume, or any combination thereof. In still other embodiments, the acute heart failure is acute decompensated heart failure without evidence of end-organ hypoperfusion or acute coronary syndrome.

In one embodiment, the method includes the step of determining that the individual has a heart rate of about 75 beats/minute and about 150 beats/minute, or about 60 bpm to about 150 bpm if the individual has been administered a beta blocker. In one embodiment, the istaroxime or pharmaceutically acceptable salt, solvate, or hydrate thereof is administered at a dose of about 1.0 μg/kg/min to about 1.5 μg/kg/min. In another embodiment, the first period of time is at least about 2 hours, or at least about 6 hours, or at least about 24 hours. In one particular embodiment, the individual is a human aged at least 18 years old. In another embodiment, the individual has at least one of dyspnea at rest or minimal exertion, congestion on chest x-ray or lung ultrasound with brain natriuretic peptide of less than about 400 μg/mL or N-terminal pro b-type Natriuretic Peptide of at least 1,400 μg/ml, or left ventricular ejection fraction of less than about 40%, or echocardiogram confirming ejection fraction of less than about 40%.

In an embodiment of the method, the one or more parameters of heart function further comprise heart rate (HR), diastolic relaxation, systolic contraction, dyspnea, chest congestion, diastolic blood pressure (DBP), systolic blood pressure (SBP), creatine clearance, deceleration slope, mitral inflow velocity, mean arterial pressure, brain natriuretic peptide levels (BNP), NT-pro-BNP levels, troponin levels, venous lactate levels, echocardiographic measurements, left ventricle end diastolic diameter (EDD), left ventricle end systolic diameter (ESD), left ventricle end diastolic volume (EDV), left ventricle end systolic volume (ESV), left atrium diameter (LAD), left atrium area (LAA), left atrium volume (LAV), E wave, A wave, E wave deceleration time (EDT), E/A ratio, Ea, Aa, E/Ea ratio, left ventricle ejection fraction (LVEF), Sa, stroke volume (SV), cardiac output (CO), stroke volume index (SVI), cardiac index (CI), pulmonary arterial systolic pressure (PASP), tricuspid annular plane systolic excursion (TAPSE), right ventricle Sa, mitral regurgitation (MR), inferior vena cava diameter (IVC), or any combination thereof. In another embodiment, the individual is undergoing a therapeutic treatment for pre-cardiogenic shock or cardiogenic shock with one or more further therapeutically active ingredients, such as, but not limited to ACE inhibitors (e.g., Lisinopril or Ramipril), ARBs (e.g., valsartan, candesartan, olmesartan, telmisartan, or losartan), diuretics (e.g., furosemide, bumetanide, torasemide, metolazone, an aldosterone antagonist, or a thiazide), Ca channel blockers, beta-blockers (e.g., carvedilol or metoprolol), digitalis, NO donors, vasodilators (e.g., hydralazine, amlodipine, felodipine, diltiazem, and verapamil), SERCA2a stimulators, neprilysin (NEP) inhibitors, myosin filament activators, recombinant relaxin-2 mediators, recombinant NP protein, activators of the soluble Guanylate Cyclase (sGC), and/or beta-arrestin ligand of Angiotensin II receptor (e.g., sacubitril). In another embodiment, the pharmaceutical composition comprises saline as the pharmaceutically acceptable carrier, and the istaroxime is reconstituted from a lyophilized powder comprising the istaroxime admixed with a bulking agent. In yet another embodiment of the method, the pharmaceutical composition comprises saline as the pharmaceutically acceptable carrier, and the istaroxime is reconstituted from a lyophilized powder comprising the istaroxime admixed with a bulking agent.

The invention also features a method of treating pre-cardiogenic shock in an individual that includes the steps of: a) providing an individual with pre-cardiogenic shock with acute heart failure, wherein the pre-cardiogenic shock comprises a systolic blood pressure of less than about 90 mm Hg without end-organ hypoperfusion or myocardial infarction; b) administering to the individual a therapeutically effective amount of a pharmaceutical composition comprising: (i) a pharmaceutically acceptable carrier; and (ii) istaroxime or a pharmaceutically acceptable salt, solvate, or hydrate thereof, wherein the istaroxime or pharmaceutically acceptable salt, solvate, or hydrate thereof is administered at a dose of about 0.2 μg/kg/min to about 2.0 μg/kg/min, and wherein the administering comprises intravenous infusion for at least about 6 hours; and c) measuring one or more parameters of heart function in the individual, wherein the one or more parameters of heart function comprises blood pressure. Further, the administering of the pharmaceutical composition results in an increase over baseline for the blood pressure, the increase measurable by 6 hours following the start of the intravenous infusion, thereby treating pre-cardiogenic shock in the individual.

In one embodiment of the method, the blood pressure is systolic blood pressure, and the administration of the pharmaceutical composition results in an increase over baseline for the systolic blood pressure. In another embodiment, the blood pressure is measured as systolic blood pressure area under the curve, and the administration of the pharmaceutical composition results in an increase over baseline for the systolic blood pressure area under the curve. In still another embodiment, the one or more parameters of heart function in the individual additionally includes cardiac index, left atrial area, stroke volume, left ventricular end-systolic volume, left ventricular end-diastolic volume, or any combination thereof. For instance, the administering of the pharmaceutical composition may result in one or more of increased cardiac index, decreased left atrial area, increased stroke volume, decreased left ventricular end-diastolic volume, and/or decreased left ventricular end-diastolic volume.

These and other features and advantages of the present disclosure will become better understood with regard to the following description, appended claims, and accompanying drawings.

### BRIEF DESCRIPTION OF THE DRAWINGS

FIG. 1 is a flowchart of an exemplary study for testing the efficacy of istaroxime for use in the treatment of pre-cardiogenic shock.

FIG. 2 is a chart showing the change in systolic blood pressure (SBP) for individuals treated with istaroxime (solid area) versus placebo (hatched area) over time. The y-axis presents the change from baseline of SBP (mmHg), whereas the x-axis represents hours from infusion (h).

FIG. 3 are graphs showing changes in blood pressure (BP) over time for individuals treated with istaroxime (solid line) versus placebo (dashed line). Panel A shows the changes in SBP up to 6 hours following infusion, while Panel B shows the changes in SBP up to 24 hours following infusion. Panel C shows the mean changes in diastolic blood pressure (DBP) up to 96 hours from infusion. The y-axis in each represents the change from baseline of BP (mmHg), whereas the x-axis represents hours from infusion.

FIG. 4 are graphs showing levels of heart and renal function parameters over time. Panel A shows changes in MAP for individuals treated with istaroxime (solid line) versus placebo (dashed line). Panel B is a graph showing the change in blood levels of eGFR over time between individuals treated with either 1.0 μg/kg/min (light gray line), 1.5 μg/kg/min (gray line), or placebo (dashed dark gray line). Panel C is a graph showing the change in blood levels of NT-proBNP over time between individuals treated with either 1.0 μg/kg/min (light gray line), 1.5 μg/kg/min (gray line), or placebo (dashed dark gray line).

### DETAILED DESCRIPTION

The compositions and methods disclosed herein provide, for the first time, a treatment regimen for individuals with pre-cardiogenic shock or cardiogenic shock. Surprisingly, individuals with pre-cardiogenic shock that were administered istaroxime exhibited a concomitant increase in both cardiac index and blood pressure (e.g., systolic blood pressure), which is unique and has not been observed with any previous intravenous drugs administered to individuals with pre-cardiogenic shock or cardiogenic shock. In some embodiments, individuals with pre-cardiogenic shock or cardiogenic shock will be administered istaroxime by intravenous infusion for a period of time (e.g., up to about 6 hours, or up to about 24 hours, or more) and exhibit an increase in systolic blood pressure by 6 hours and/or by 24 hours as compared to similarly-situated individuals (i.e., with pre-cardiogenic shock or cardiogenic shock) who were not administered istaroxime or were administered a placebo. Such individuals who are administered istaroxime may exhibit one or more additional cardiac improvements, such as, but not limited to, increased cardiac index, decreased left atrial area, decreased left ventricular end-systolic volume, and decreased left ventricular end-diastolic volume as compared to such individuals prior to administration of istaroxime and/or compared to individuals with pre-cardiogenic shock or cardiogenic shock that are not administered istaroxime.

Provided herein are compositions comprising istaroxime suitable for administration to individuals having pre-cardiogenic shock or cardiogenic shock symptoms. In one particular embodiment, istaroxime is administered to these individuals by intravenous infusion for up to 24 hours to increase systolic blood pressure and alleviate symptoms of pre-cardiogenic shock or cardiogenic shock. For instance, in one embodiment, istaroxime is administered by intravenous infusion to an individual presenting with pre-cardiogenic shock without evidence of end-organ hypoperfusion or acute coronary syndrome to treat pre-cardiogenic shock and prevent progression to overt cardiogenic shock.

The compositions and methods disclosed herein will be described in more detail below.

### Definitions

Unless defined otherwise, all technical and scientific terms used herein have the same meaning as those commonly understood by one of ordinary skill in the art to which this invention belongs. Standard techniques are used unless otherwise specified. Although methods and materials similar or equivalent to those described herein can be used in the practice or testing of the present disclosure, suitable methods and materials are described below. The materials, methods and examples are illustrative only, and are not intended to be limiting. All publications, patents and other documents mentioned herein are incorporated by reference in their entirety.

As used herein, the singular forms “a,” “an,” and “the” include the plural referents unless the context clearly indicates otherwise.

The term “about” refers to the variation in the numerical value of a measurement, e.g., volume, time, pressure, concentration, etc., due to typical error rates of the device used to obtain that measure. In one embodiment, the term “about” means within 5% of the reported numerical value, preferably, the term “about” means within 3% of the reported numerical value.

The term “cardiogenic shock” as used herein refers to a condition in an individual, such as a human patient, where the heart cannot pump enough blood and oxygen to the brain, kidneys, and other vital organs leading to end-organ hypoperfusion. Overt “cardiogenic shock” or SCAI Stage C “cardiogenic shock” is defined by a systolic blood pressure of less than about 90 mmHg (hypotension), a cardiac index of less than about 2.2 L/min/m², pulmonary capillary wedge pressure of greater than 15 mmHg, and evidence of end-organ hypoperfusion. “Pre-cardiogenic shock”, SCAI Stage B “pre-cardiogenic shock”, or “early cardiogenic shock” refer to a condition where the individual has not yet developed overt “cardiogenic shock” and is defined by a systolic blood pressure of less than about 90 mmHg without evidence of end-organ hypoperfusion.

The term “heart failure” refers to a clinical syndrome characterized by typical symptoms (e.g., breathlessness, ankle swelling and fatigue) that may be accompanied by signs (e.g., elevated jugular venous pressure, pulmonary crackles and peripheral edema) caused by a structural and/or functional cardiac abnormality, resulting in a reduced cardiac output and/or elevated intracardiac pressures at rest or during stress.

The terms “acute heart failure” or “AHF” are used interchangeably herein and refer generally to a rapid onset or worsening of symptoms and/or signs of HF requiring immediate treatment and hospitalization. The current definition of “acute heart failure” is rather nonspecific and may include a broad spectrum of conditions with several phenotypes characterized by different clinical presentation, etiology, precipitating factors, therapeutic approach, and prognosis. In addition, a large proportion of human patients have a subacute course of the disease with a progressive worsening of signs and symptoms of HF which could develop days before hospital admission.

The term “acute decompensated heart failure” as used herein refers to a sudden worsening of the signs and symptoms of heart failure, which, in humans, typically includes difficulty breathing (dyspnea), leg or feet swelling, and fatigue.

The term “acute coronary syndrome” as used herein refers to a sudden reduction or blockage of blood flow to the heart. A common example of “acute coronary syndrome” is a myocardial infarction (heart attack).

The terms “chronic heart failure” or “CHF” are used interchangeably herein and refer to the current clinical classification of chronic HF based on the presence of signs and symptoms of HF and left ventricular ejection fraction (LVEF) in an individual, such as a human patient, recognizing three categories: “heart failure with reduced ejection fraction” or “HFrEF,” which is characterized by an LVEF of less than about 40%; “heart failure with mid-range ejection fraction” or “HFmEF” or “HFmrEF,” which is characterized by an LVEF from about 40% to about 49%; and “heart failure with preserved ejection fraction” or “HFpEF,” which is characterized by an LVEF of equal to or greater than about 50%. The terms “HFmrEF” and “HFpEF” include two additional criteria, namely increased natriuretic peptides levels (BNP >35 μg/ml and/or NT-proBNP >125 μg/mL) associated with the evidence of structural and/or functional heart disease (left ventricular hypertrophy and/or left atrium enlargement and/or evidence of diastolic dysfunction).

The term “hypotension” as used herein is defined as systolic blood pressure (SBP) that is less than 90 mmHg or mean arterial blood pressure (MAP) less than about 60 mmHg or greater than 30 mmHg drop from baseline.

The term “end-organ hypoperfusion” as used herein means clinical signs such as cold, clamped extremities, poor urine output, mental confusion, elevated lactate or increased creatinine.

The term “treating” refers to any indicia of success in the treatment or amelioration of the disease or condition in an individual. Treating can include, for example, reducing or alleviating the severity of one or more symptoms of the disease or condition, or it can include reducing the frequency with which symptoms of a disease, defect, disorder, or adverse condition, and the like, are experienced by an individual, such as a human patient.

The term “preventing” refers to the prevention of the disease or condition, e.g., pre-cardiogenic shock or cardiogenic shock, in an individual, such as a human patient. For example, if an individual at risk of developing pre-cardiogenic shock or cardiogenic shock is treated with the methods of the present invention and does not later develop pre-cardiogenic shock or cardiogenic shock, then the disease has been prevented in that individual.

The term “treat or prevent” is sometimes used herein to refer to a method that results in some level of treatment or amelioration of the disease or condition, and contemplates a range of results directed to that end, including but not restricted to prevention of the condition entirely.

As used herein, the term “pharmaceutically acceptable” salt, solvate, hydrate, or ester means a salt, solvate, hydrate, or ester form of the active ingredient which is compatible with any other ingredients of the pharmaceutical composition, which is not deleterious to the subject to which the composition is to be administered.

As used herein, the term “pharmaceutically acceptable carrier” means a chemical composition with which an istaroxime compound may be combined and which, following the combination, can be used to administer the compound to a mammal.

The term “intravenous infusion” refers to the administration or delivery of liquid substances directly into a vein of a mammal. Typical “infusions” use only the pressure supplied by gravity.

The term “parameter” as used herein to refer to measuring heart function means any heart function that is observable or measurable using suitable measuring techniques available in the art. A non-limiting list of exemplary “parameters” of heart function include heart rate (HR), blood pressure (BP), diastolic relaxation, systolic contraction, dyspnea, chest congestion, diastolic blood pressure (DBP), systolic blood pressure (SBP), systolic blood pressure area under the curve (SBP AUC), creatine clearance, deceleration slope, mitral inflow velocity, mean arterial pressure, brain natriuretic peptide levels (BNP), NT-pro-BNP levels, troponin levels, venous lactate levels, echocardiographic measurements, left ventricle end diastolic diameter (EDD), left ventricle end systolic diameter (ESD), left ventricle end diastolic volume (EDV), left ventricle end systolic volume (ESV), left atrium diameter (LAD), left atrium area (LAA), left atrium volume (LAV), E wave, A wave, E wave deceleration time (EDT), E/A ratio, Ea, Aa, E/Ea ratio, left ventricle ejection fraction (LVEF), Sa, stroke volume (SV), cardiac output (CO), stroke volume index (SVI), cardiac index (CI), pulmonary arterial systolic pressure (PASP), tricuspid annular plane systolic excursion (TAPSE), right ventricle Sa, mitral regurgitation (MR), inferior vena cava diameter (IVC), and the like. As one having ordinary skill in the art will appreciate, measuring one or more “parameters” of heart function can be used to detect heart dysfunction as compared to the average normal parameters and can also be used to determine whether heart function has improved following or during treatment.

The terms “therapeutically active” or “active” ingredient or compound refer to a substance that provides a beneficial effect to the individual to whom the substance is administered. A “therapeutically effective amount” or “therapeutically effective dose” is the amount of a composition or active ingredient sufficient to provide a beneficial effect to the individual to whom the composition or active ingredient is administered.

### DESCRIPTION

The present invention is directed to administration of istaroxime to individuals, such as human patients, to treat or prevent pre-cardiogenic shock or cardiogenic shock. In a preferred embodiment, the route of administration of istaroxime is by intravenous infusion. For individuals exhibiting symptoms of pre-cardiogenic shock, istaroxime infusion is administered to treat the pre-cardiogenic shock (SCAI Stage B) and prevent progression to overt cardiogenic shock. In some embodiments, istaroxime infusion can be administered to individuals presenting with overt cardiogenic shock (SCAI Stage C) to alleviate the symptoms and improve heart function.

In one embodiment, the compositions and treatment methods alleviate symptoms associated with pre-cardiogenic shock or cardiogenic shock in individuals with acute decompensated heart failure (ADHF), which is an acute decompensation of an underlying chronic systolic heart failure due to, e.g., arterial hypertension, ischemic heart disease, or dilated cardiomyopathy. In some embodiments, the pre-cardiogenic shock or cardiogenic shock is not caused by an acute coronary syndrome, such as a myocardial infarction. Individuals with pre-cardiogenic shock or early cardiogenic shock may present with persistent hypotension characterized by a systolic blood pressure (SBP) of less than about 90 mmHg without evidence of an acute coronary syndrome and without evidence of end-organ hypoperfusion. Therefore, administration of istaroxime by intravenous infusion to these individuals raises the SBP thereby alleviating the hypotension associated with pre-cardiogenic shock and preventing progression to SCAI Stage C overt cardiogenic shock. In other cases, individuals with pre-cardiogenic shock or early cardiogenic shock may present as normotensive with evidence of end-organ hypoperfusion and without evidence of an acute coronary syndrome.

In some embodiments, the istaroxime infusion is between about 3 hours and up to 24 hours or more, e.g., 3 h, 4 h, 5 h, 6 h, 7 h, 8 h, 9 h, 10 h, 11 h, 12 h, 13 h, 14 h, 15 h, 16 h, 17 h, 18 h, 19 h, 20 h, 21 h, 22 h, 23 h, 24 h, or more hours. For instance, the infusion can be for up to 24 hours, or longer. In some embodiments, the duration of the infusion is from about 6 hours to about 24 hours, or from about 12 hours to about 24 hours, or about 18 hours to about 24 hours. In other embodiments, the duration of the infusion is greater than about 24 hours, e.g., 25 h, 26 h, 27 h, 28 h, 29 h, 30 h, 31 h, 32 h, 33 h, 34 h, 35 h, 36 h, 37 h, 38 h, 39 h, 40 h, 41 h, 42 h, 43 h, 44 h, 45 h, 46 h, 47 h, 48 h, 54 h, 60 h, 66 h, 72 h, 78 h, 84 h, 90 h, 96 h, 5 days, 6 days, 7 days, 8 days, 9 days, 10 days, 11 days 12 days, 13 days, 14 days, or longer. It being understood that the infusion of istaroxime provides an improvement in the heart function of the individual, e.g., an increase in SBP, within about 24 hours from the start of infusion; preferably, the improvement occurs within about 12 hours from the start of infusion, or within about 6 hours from the start of the infusion, or within about 3 hours from the start of infusion, or within about 1 hour from the start of infusion. For instance, in one embodiment, an individual presenting with pre-cardiogenic shock or cardiogenic shock who is administered istaroxime exhibits an improvement in SBP within 6 hours after the start of infusion as compared to an individual presenting with pre-cardiogenic shock or cardiogenic shock who is administered a placebo.

Istaroxime is an inotropic compound having the following structural formula (I):

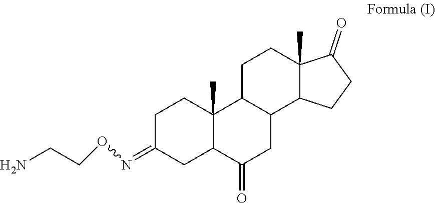 [SMILES: C[C@]12CCC(=NOCCN)CC1C(=O)CC1C2CC[C@]2(C)C(=O)CCC12]

Therefore, it is an object of the present invention to utilize istaroxime, or its pharmaceutically acceptable salt or ester, hydrate, solvate, or polymorphic form in a medicament to treat or prevent pre-cardiogenic shock or cardiogenic shock in an individual. In a preferred embodiment, istaroxime is administered to an individual for the treatment of pre-cardiogenic shock and to prevent the worsening of the individual's condition to overt cardiogenic shock.

Also disclosed herein is a pharmaceutical composition comprising istaroxime in an admixture with at least one pharmaceutically acceptable vehicle and/or excipient. In preferred embodiments, the pharmaceutical composition is formulated for administering to an individual by infusion, preferably, it is by intravenous infusion.

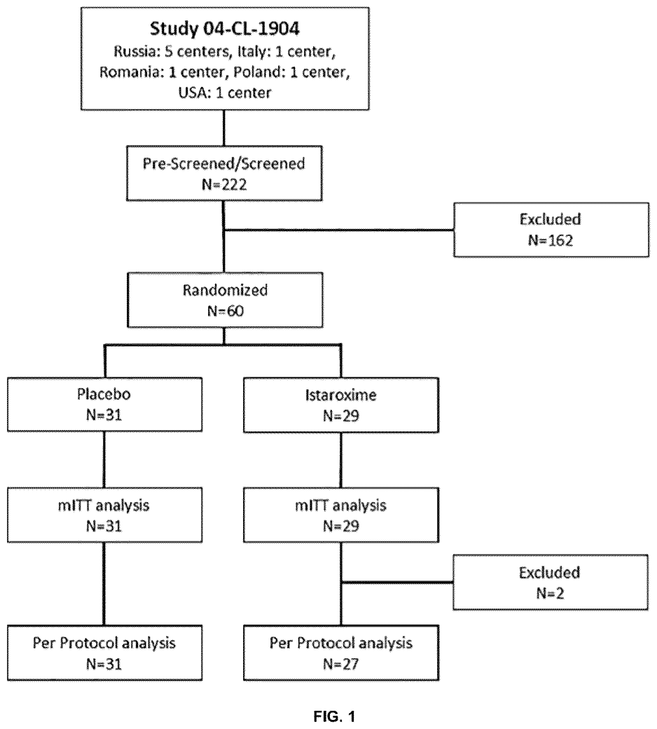

The efficacy of the present invention for the treatment of pre-cardiogenic shock can be assessed in the exemplary clinical study summarized in Table 1 below and FIG. 1.

TABLE 1
Exemplary Clinical Study

| Title | A Multicenter, Randomized, Double-Blind, Placebo-Controlled, Parallel Group | | |
|---|---|---|---|
| | Study on the Safety and Efficacy of Istaroxime for Pre-Cardiogenic Shock | | |
| | (SEISMIC) | | |
| Indication | Pre-cardiogenic shock SCAI stage B caused by ADHF without evidence for an | | |
| | acute coronary syndrome. | | |
| Objective | To assess the safety, tolerability, and efficacy of istaroxime to increase systolic | | |
| | blood pressure (SBP) in patients with pre-cardiogenic shock or cardiogenic shock, | | |
| | defined as hospitalization for ADHF with persistent hypotension (SBP 75-90 | | |
| | mmHg for two hours) who, for at least 6 hours prior to Screening, are not on | | |
| | cardiovascular, respiratory, or renal mechanical support, and have not received | | |
| | intravenous vasopressors or inotropes. | | |
| Study Design | A multicenter, randomized, double-blind, placebo-controlled, parallel group | | |
| | study. | | |
| Study Period | The study includes a screening period (Days 0-1), a treatment period (Day 1), a | | |
| | post-treatment period (Days 2-5), and a follow up period (Days 6-30). Subject | | |
| | participation will be from screening to Day 30 or death, whichever occurs first. | | |
| Subject Selection | Inclusion Criteria | | |
| Criteria | Subjects who fulfill the following inclusion criteria at screening will be considered | | |
| | for the study: | | |
| | 1. | Clinical presentation consistent with SCAI Stage B pre-cardiogenic shock | |
| | caused by acute decompensation of chronic systolic heart failure (due to arterial | | |
| | hypertension, ischemic heart disease or dilated cardiomyopathy), without | | |
| | evidence for an acute coronary syndrome. | | |
| | 2. | Signed informed consent form (ICF); | |
| | 3. | Males and females, 18 to 85 years of age (inclusive); | |
| | 4. | An admission within 36 hours prior to randomization for ADHF episode, | |
| | defined as: | | |
| | | a. | Dyspnea, at rest or with minimal exertion, |
| | | b. | Congestion on chest x-ray or lung US with BNP ≥400 pg/mL or |
| | | | NT-proBNP ≥1400 pg/mL. |
| | | Elective admissions for medications tune up or procedures do not qualify | |
| | | as an ADHF admission. | |
| | 5. | History of left ventricular ejection fraction (LVEF) <40%; | |
| | 6. | Persistent hypotension defined as: | |
| | | a. | SBP between 75 and 90 mmHg for at least 2 hours prior to |
| | | | Screening; |
| | | b. | SBP does not decrease by >7 mmHg on two separate measurements |
| | | | during the last 2 hours prior to randomization; |
| | 7. | Heart rate 75 to 150 bpm. If the subject is on a beta-blocker, the | |
| | range is 60 to 150 bpm; and | | |
| | 8. | Echocardiogram during index hospitalization confirming ejection fraction <40% | |
| | and no evidence of other pathology to confound interpretation of cardiac | | |
| | physiology (e.g., pericardial effusion). | | |
| | Exclusion Criteria | | |
| | Subjects meeting any of the following exclusion criteria would be considered | | |
| | ineligible for this study: | | |
| | 1. | Cardiogenic shock of SCAI stage C or worse; | |
| | 2. | Cardiogenic shock due to any other condition besides acute | |
| | decompensation of chronic heart failure. | | |
| | 3. | Any of the following in the past 30 days: acute coronary syndrome, | |
| | coronary revascularization, MI, CABG, or percutaneous coronary intervention; | | |
| | 4. | Current treatment (within 6 hours of Screening) with positive inotropic | |
| | agents or vasopressors, renal support including ultrafiltration, or mechanical | | |
| | circulatory, ventilatory or renal support (intra-aortic balloon pump, endotracheal | | |
| | intubation, mechanical ventilation, or any ventricular assist device); | | |
| | 5. | Venous Lactate >2 mmol/L; | |
| | 6. | History of heart transplant or UNOS priority la heart transplant listing; | |
| | 7. | Ongoing treatment with digoxin (if digoxin was stopped before signing | |
| | the ICF and the digoxin plasma level is <0.5 ng/ml, the patient may be enrolled); | | |
| | 8. | Severe renal impairment (eGFR <30 ml/min, calculated by the MDRD | |
| | formula); | | |
| | 9. | Hypersensitivity to the study medication and its excipients (including | |
| | known lactose hypersensitivity) or any related medication; | | |
| | 10. | Stroke or TIA within 3 months; | |
| | 11. | Incomplete revascularization (patients with ischemic heart disease have to | |
| | have had a catheterization in the last year demonstrating that the main coronary | | |
| | arteries are well revascularized); | | |
| | 12. | Any significant valvular disease (including moderate or severe valvular | |
| | disease, such as severe aortic stenosis or regurgitation); severe tricuspid or mitral | | |
| | regurgitation; | | |
| | 13. | Primary hypertrophic or restrictive cardiomyopathy or systemic illness | |
| | known to be associated with infiltrative heart disease; | | |
| | 14. | Admission for AHF triggered primarily by a correctable etiology such as | |
| | significant arrhythmia (inclusive of atrial fibrillation as the main reason for | | |
| | admission), infection, severe anemia, acute coronary syndrome, pulmonary | | |
| | embolism, exacerbation of COPD, planned admission for device implantation, or | | |
| | over-diuresis as a cause of hypotension; | | |
| | 15. | Pericardial constriction or active pericarditis; | |
| | 16. | Life-threatening ventricular arrhythmia or implantable cardioverter | |
| | defibrillator (ICD) shock within the past month or history of sudden death within | | |
| | 6 months; | | |
| | 17. | Cardiac resynchronization therapy (CRT), ICD, or pacemaker | |
| | implantation within the past month; | | |
| | 18. | Sustained ventricular tachycardia in the last 3 months with no | |
| | defibrillator; | | |
| | 19. | Cor pulmonale or other causes of isolated right-sided HF or not related to | |
| | left ventricular dysfunction; | | |
| | 20. | Acute respiratory distress syndrome; | |
| | 21. | Suspected sepsis; fever >38° or active infection requiring IV | |
| | antimicrobial treatment; | | |
| | 22. | Body weight <40 kg or ≥150 kg; | |
| | 23. | Laboratory exclusions: | |
| | | a. | Hemoglobin <9 g/dl, |
| | | b. | Platelet count <100,000/μl, |
| | | c. | Serum potassium >5.3 mmol/l or <3.5 mmol/l; |
| | 24. | A life expectancy <3 months in the opinion of the investigator; | |
| | 25. | Severe pulmonary or thyroid disease; | |
| | 26. | Pregnant or breast-feeding; | |
| | 27. | Ongoing drug or alcohol abuse; | |
| | 28. | Participation in another interventional study within the past 30 days. | |
| Study Drugs | Test drug: Istaroxime lyophilized powder (10 mg plus 50 mg lactose per vial) | | |
| | Comparator (placebo): 50 mg lactose lyophilized powder | | |
| Mode | Intravenous infusion via a syringe pump. | | |
| Administrator | | | |
| Treatment | Treatment by intravenous infusion will last up to 24 hours | | |
| Duration | | | |

| Dosing Scheme | Istaroxime 1.0-1.5 mg/kg/min since the beginning. A continuous intravenous | |
|---|---|---|
| | infusion for 24 hours. | |
| Sample Size | 60 individuals | |
| Study Procedures | Screening period (between Hours −24 to −1) | |
| | Within a maximum of 24 hours before administration of study medication | |
| | (istaroxime), a medical screening will be performed on all prospective patients to | |
| | assess suitability for the study. Prior to conducting any study specific procedures, | |
| | the investigator or his/her designee will explain the study fully to the patient and | |
| | provide him/her with a copy of the Patient Information Sheet and Informed Consent | |
| | Document. If the patient is willing to participate in the study, s/he and the | |
| | investigator or his/her designee will both sign the Informed Consent Document and | |
| | a copy of the signed document will be kept by the patient. | |
| | Treatment period and post Treatment Period (Day 1 to Day 3) | |
| | 1) | Confirm eligibility; |
| | 2) | Randomization of patients (after eligibility has been confirmed); |
| | 3) | Within 15 minutes of infusion start, assess vital signs, cTn(T or I), NT- |
| | | proBNP or BNP, ECG, EQ-5D, physician assessment of heart failure, |
| | | invasive hemodynamic measures, and venous lactate; |
| | 4) | Obtain blood samples for centrally-analyzed biomarkers and |
| | | pharmacokinetics and start Holter monitoring (for 72 hours); |
| | 5) | Insertion of multiple lumen intravenous catheter; |
| | 6) | Start istaroxime or placebo infusion (date and time of infusion start must |
| | | be recorded in the case report form); |
| | 7) | Measure blood pressure and heart rate at 0.5, 1, 2 hours after start of |
| | | infusion, and then hourly to 24 hours after start of infusion, and then at |
| | | 30, 36, 48, 60, 72, 84, and 96 hours as well as Day 30; |
| | 8) | Measure temperature at 24, 48, and 72 hours after start of infusion; |
| | 9) | Measure oxygen saturation at 1, 2, 3, 4, 5, 6, 12, 18, 24, 30, 36, 48, 60, |
| | | and 72 hours, and Day 30 after start of infusion; |
| | 10) | Measure body weight at 24, 48, 72, and 96 hours, and Day 30 after start |
| | | of infusion; |
| | 11) | Collect urine output at 0-24 and 24-48 hours after start of infusion; |
| | 12) | cTn(T or I), NT-proBNP, BNP, venous lactate assessment at 12, 24, 48, |
| | | 72, and 96 hours after start of infusion; |
| | 13) | Perform daily laboratory tests for chemistry (electrolytes, liver function |
| | | tests, lipids, creatine, urea, BUN, glucose, albumin, protein, eGFR) and |
| | | hematology at 24, 48, 72, and 96 hours, and Day 30 after start of infusion; |
| | 14) | Continue 24-hour Holter electrocardiogramand arrythmia monitoring |
| | | for 72 hours after start of infusion, and record 12-lead electrocardiogram |
| | | at 12, 24, 48, 72, and 96 hours, and Day 30 after start of infusion; |
| | 15) | Collect invasive hemodynamic measurements for patients with a Swan |
| | | Ganz catheter at 3, 6, 12, 24, and 30 hours after start of infusion; |
| | 16) | Evaluate LVEDD, LVESD, LVEDV, LVESV, LVEF, 2D global |
| | | longitudinal strain, LAD, LAA, LAV, mitral regurgitation, ERO, CI, SVI, |
| | | ejection time, VTI, LVOT, E, A, E/A ratio, Sa, Ea, E/Ea ratio, PAPs, |
| | | TAPSE, right ventricle Sa, inferior VCD, and CO at 24 and 30 hours after |
| | | start of infusion; |
| | 17) | Draw blood samples for pharmacokinetic profile assessment at 6, 12, and |
| | | 24 hours after start of infusion, and at 0.25, 0.5, 1, 6, 12, and 24 hours after |
| | | the end of infusion; |
| | 18) | Conduct physical examination at 24, 48, 72, and 96 hours, and Day 30 |
| | | after start of infusion; |
| | 19) | Concomitant medication monitoring (including chronic medication; dose, |
| | | date and time must be recorded on case report form); |
| | 20) | Conduct worsening HF assessment at 24, 48, 72, and 96 hours after start |
| | | of infusion; |
| | 21) | Completion of EQ-5D quality of life questionnaire by patient at 96 hours |
| | | and Day 30; |
| | 22) | Monitor adverse events; and |
| | 23) | Report readmission and death through patient discharge until Day 30. |
| Efficacy | Efficacy endpoints | |
| Parameters | 1. | Primary efficacy endpoint: |
| | | Change from baseline in SBP AUC at 6 hours (SBP_{AUC(0-6)}) measured via a |
| | | sphygmomanometer or arterial line. |
| | 2. | Secondary efficacy endpoints: |
| | | Treatment-failure score, based on death, circulatory, respiratory, or renal |
| | | mechanical support or intravenous inotrope or vasopressor treatment, and |
| | | changes in systolic blood pressure; |
| | | Change from baseline in SBP at 6 and 24 hours of treatment, measured via |
| | | a sphygmomanometer or arterial line; |
| | | SBP AUC to 24 hours from infusion start (SBP_{AUC(0-24)}); |
| | | Number of subjects with increases from baseline in SBP ≥5% and ≥10 |
| | | mmHg at a timepoint between 4-6 hours after dosing and at least one other |
| | | measurement separated by ≥2 hours during the 24 hours infusion; |
| | | Number of subjects requiring treatment with intravenous vasopressors, |
| | | inotropes, and/or mechanical cardiac or renal support or have died from |
| | | randomization to 24 hours and Day 5 (“treatment failure”). |
| | | Changes in quality of life measured by the EQ-5D from baseline to Day 5 |
| | | (96 hours) from infusion start and at Day 30; |
| | | Change from baseline in creatinine clearance at 24, 48, 72 and 96 hours |
| | | from infusion start; |
| | | Change from baseline and observed heart rate measurements at 12, 24, 48, |
| | | 72 and 96 hours from infusion start; |
| | | Change from baseline and observed mean arterial pressure (MAP) at 12, |
| | | 24, 48, 72 and 96 hours from infusion start; |
| | | Change from baseline and observed brain natriuretic peptide (BNP), NT- |
| | | pro-BNP, troponin (cTn; either T or I) and venous lactate at 12, 24, 48, 72 |
| | | and 96 hours from infusion start; |
| | | Time to worsening heart failure through Day 5; |
| | | Time to HF re-admission or death through Day 30; |
| | | Length in ICU/length of initial hospitalization; |
| | | Days alive and out of acute care (including all intensive acute care units); |
| | | Days alive and out of the hospital through Day 30; |
| | | Number of subjects with hospital re-admissions through Day 30; |
| | | Mortality and reasons for death through Day 30; |
| | | For patients who are invasively monitored (pulmonary artery catheter) - |
| | | changes in invasive hemodynamic parameters from pre-treatment to 3, 6, |
| | | 12, 24 and 30 hours; and |
| | | Echocardiographic measurements at 24 and 30 hours. Changes in |
| | | measurements will be assessed when data is available. |

| | |
|---|---|
| Safety Parameters | Safety endpoints: |

| Tolerability of Drug Administration: | |
|---|---|
| 1. | Site infusion pain; |
| 2. | Site administration pain; and |
| 3. | Collect urine output at 0-24 and 24-48 hours after start of |
| | infusion; |

Incidence of adverse events (AEs) and serious AEs (SAEs); and
Clinically significant arrythmias during the infusion and for 48 hours after
the infusion has been stopped.

| | |
|---|---|
| Statistical Analysis | Sample Size Determination: |

For the primary endpoint (SBP AUC₀₋₆) in the active group, it is assumed
that the change in SBP will be from 0 to 3 mmHg at 1 hour, to 6 mmHg
at 2 hours, and to 10 mmHg at 3 hours, where it will remain steady
through 6 hours. In placebo, it is assumed that the change in SBP will be
from 0 to 2 mmHg at 1 hour, to 3 mmHg at 3 hours, where it will remain
steady through 6 hours;
With these assumptions, the AUCs are 44 mmHg · h for active and 15.25
mmHg · h for placebo. Assuming the SD of 14.5 mmHg · h is the same at
all time points and the same in both groups, and there are no dropouts,
the SD for the AUC is approximately 5.7 mmHg · h. Therefore, with 30
subjects/group we would have >99% power to detect the assumed
difference in AUCs at a 1-sided α of 0.025;
Therefore, 60 subjests will be enrolled.

Primary efficacy endpoint:

The primary efficacy endpoint is the SBP area under the change from
baseline curve from baseline through hour 6 following the start of infusion
(AUC₀₋₆), computed by trapezoidal rule after applying any applicable
imputations;
The primary analysis population will be the mITT population set; values
after an intercurrent event (e.g., death, infusion stopped due to an AE) will
be set to missing. The mean AUC₀₋₆ will be compared between treatment
groups using analysis of covariance (ANCOVA) with pooled site,
treatment, and baseline value in the model;
An additional analysis will be conducted to examine composite endpoints
that incorporate the intercurrent events through truncation of the
distribution of AUC₀₋₆. For this analysis, the AUC₀₋₆ for patients with a
value less than −5 mmHg · h, who die, or who require rescue therapy will be
set to −5 mmHg · h. Treatment groups will be compared using a t-test. The
medians in each treatment group, which are invariant to the truncation, will
be presented; and
In addition, an analysis where the AUC₀₋₆ for patients with a value less
than −5 mmHg · h, who die, or who require rescue therapy will be set to 0
mmHg · h (instead of −5 mmHg · h). Treatment groups will be compared
using ANCOVA adjusted for baseline SBP. The medians in each treatment
group, which are invariant to the truncation, will be presented.

Secondary efficacy endpoint:

Changes from baseline in SBP at 6 and 24 hours will be compared between
treatment groups using a mixed-effects repeated measurement model
(MMRM) with baseline value, pooled site, treatment, visit, and treatment x
visit in the model.
The area under the change from baseline curve from baseline through hour
24 (AUC₀₋₂₄) will be computed by trapezoidal rule after applying any
applicable imputations, as above for the primary endpoint. Treatment
groups will be compared using an ANCOVA model with adjustment for
pooled study site and baseline SBP. In addition, a composite endpoint that
incorporates intercurrent events will be computed. The treatment effect
estimated will reflect the probability of a better clinical response with
respect to blood pressure with istaroxime treatment. A rank-based approach
will be used: patients who died will be assigned the worst rank, patients
who required rescue therapy will be assigned the next worse rank, and the
remaining patients will be ranked by their AUC₀₋₂₄ (following recursive
imputation for missing values). Treatment groups will be compared using
a van Elteren's test stratified by pooled study center.
The proportion of subjects who will sustain treatment failure will be
summarized and compared using the Cochran-Mantel-Haenszel test,
controlling for pooled sites.
Changes in quality of life from baseline to 5 and 30 days will be assessed
using the EQ-5D-5L instrument and summarized by treatment group. Index
values and visual analog scale (VAS) scores will be compared between
treatments at each time point using ANCOVA with treatment and pooled
study site in the model.
Changes in eGFR from baseline to 24, 48, 72 and 96 hours will be
compared between treatment groups using a MMRM with baseline eGFR,
treatment, pooled study site, visit, and treatment x visit in the model.
The change from baseline and observed values of heart rate, MAP,
natriuretic peptides (BNP, NT-proBNP), troponin (cTn), and venous lactate
will be presented at each time point. cTn, BNP, and NT-proBNP will be log-
transformed for analysis. The changes from baseline to 12, 24, 48, 72 and
96 hours will be compared between treatment groups using a MMRM for
each outcome that includes baseline value, treatment, pooled study site,
visit, and treatment x visit in the model.
The time to the first occurrence of WHF, HF hospitalization or death
through Day 30, the time to the first HF readmission through Day 30, time
to any hospital admission through Day 30 and the time to death through
Day 30 will be compared between treatment groups using log-rank tests
stratified by pooled study center. Event times for non-fatal events will be
censored for those patients for patients without an event or at the time of
last follow-up or at the time of death.
Length of stay will be defined as the index hospitalization discharge date
minus the randomization date plus one. Subjects still in the hospital at Day
30 will be truncated at Day 30. Subjects who die during the initial
hospitalization will be assigned a value of 31. The days during the index
hospitalization spent in the ICU/CCU will be recorded as days by the
investigator. Days alive and out of hospital will be computed as 31 days
minus the days in-hospital through Day 30 (including the index
hospitalization and any rehospitalizations) minus the days following a
death prior to Day 30. Days in hospital will be computed for the index
hospitalization as the discharge date minus the baseline date plus one, and
for rehospitalizations as the discharge date minus the admission date plus
one. Treatment groups will be compared using van Elteren's tests stratified
by pooled study center.

To determine efficacy of istaroxime administration in an individual with pre-cardiogenic or cardiogenic shock, there are a number of parameters of heart function that can be measured to determine whether or not the condition has been improved or prevented. In preferred embodiments, SBP is measured using art-standard equipment, such as, but not limited to, a sphygmomanometer or an arterial line. In one particular embodiment, the measured heart function parameter is SBP area under the curve (SBP AUC), and is determined by multiplying the SBP values or change in SBP values over a particular period of time. SBP can be monitored/measured using any art standard techniques, such as, but not limited to, a sphygmomanometer or an arterial line. In particular, an arterial line, which is a thin, flexible tube that is inserted into an artery (e.g., in the radial or ulnar artery of the wrist, brachial artery of the elbow, femoral artery in the groin, or dorsalis pedis artery of the foot) to measure blood pressure in real time via a connection to a pressure transducer. ABP AUC is typically measured from baseline (time 0) to a particular endpoint, e.g., 1 h, 2 h, 3 h, 6 h, 12 h, 18 h, or 24 h after the start of istaroxime administration. For instance, SBP AUC₍₀₋₆₎ or SBP AUC₍₀₋₂₄₎ is measured. An improvement in SBP AUC within this time can be considered treating pre-cardiogenic shock or preventing overt cardiogenic shock in an individual. Further, an increase in the change in SBP after infusion, e.g., 10 min, 20 min, 30 m, 40 min, 50 min, 1 h, 1.5 h, 2 h, 3 h, 4 h, 5 h, 6 h, 7 h, 8 h, 9 h, 10 h, 11 h, 12 h, 18 h, 24 h, or more after start of istaroxime administration can be considered treating pre-cardiogenic shock or preventing overt cardiogenic shock in an individual.

In another embodiment, echocardiography is performed on individuals according to international standards [see, for example, Lang et al., 2005, J. Am. Soc. Echocardiogr. 18 (12): 1440-1463; Negueh et al., 2009, Eur. J. Echocardiogr. 10 (2): 165-193; Evangelista et al., 2008, Eur. J. Echocardiogr. 9 (4): 438-448]. Echocardiography is within the purview of skilled physicians or sonographers. For instance, echocardiography can be performed during screening of potential clinical study participants or upon admission of an individual to a health care facility. In other embodiments, echocardiography is done at baseline or 1 h, 2 h, 3 h, 4 h, 5 h, 6 h, 7 h, 8 h, 9 h, 10 h, 11 h, 12 h, 13 h, 14 h, 15 h, 16 h, 17 h, 18 h, 19 h, 20 h, 21 h, 22 h, 23 h, 24 h, 25 h, 26 h, 27 h, 28 h, 29 h, 30 h, 31 h, 32 h, 33 h, 34 h, 35 h, 36 h, 37 h, 38 h, 39 h, 40 h, 41 h, 42 h, 43 h, 44 h, 45 h, 46 h, 47 h, 48 h, 72 h, 96, h, 5 days, 6 days, 7 days, 8 days, 9 days, 10 days, 11 days, 12 days, 13 days, 14 days, 15 days 16 days 17 days, 18 days 19 days, 20 days, 21 days 22 days, 23 days, 24 days, 25 days, 26 days, 27 days, 28 days, 29 days, or 30 days following the start of infusion. In one embodiment, echocardiography is monitored during infusion and values are recorded at 24 and 30 hours post-infusion. In other embodiments, echocardiographic values are recorded 12, 24, 48, 72, and 96 hours post-infusion. Table 2 summarizes exemplary echocardiographic and other heart function parameters.

TABLE 2
Heart Function Parameters

| | |
|---|---|
| Parameter | Definition |

Cardiac dimension measures

| | |
|---|---|
| Left ventricle end | measured with M-mode echocardiography at the level of mitral |
| diastolic diameter (EDD) | valve (MV) leaflets from parasternal long axis view (PLAX) |
| | (NR: 42-59 mm human males and 39-53 mm human females) |
| Left ventricle end | measured with M-mode echocardiography at the level of mitral |
| systolic diameter (ESD) | valve (MV) leaflets from PLAX (NR: 25-35 mm) |
| Left ventricle end | measured with M-mode echocardiography at the level of mitral |
| diastolic volume (EDV) | valve (MV) leaflets from PLAX (NR: 67-155 mL human males |
| | and 56-104 mL human females) |
| Left ventricle end | measured with M-mode echocardiography at the level of mitral |
| systolic volume (ESV) | valve (MV) leaflets from PLAX (NR: 22-58 mL human males |
| | and 19-49 mL human females) |
| Left atrium diameter | measured at end-ventricular systole with M-mode |
| (LAD) | echocardiography from PLAX. (NR: 30-40 mm human males |
| | and 27-38 mm human females) |
| Left atrium area (LAA) | measured from apical four chamber view (NR: ≤20 cm2) |
| Left atrium volume (LAV) | derived from area-length measured from apical four chamber |
| | view (NR: 18-58 mL human males and 22-52 mL human |
| | females) |

Left ventricle diastolic function parameters

| | |
|---|---|
| E wave | measured from mitral valve pulsed wave Doppler, is the peak |
| | velocity of early filling. Normal range for all the diastolic |
| | parameters significantly changes with age. |
| A wave | measured from mitral valve pulsed wave Doppler is the peak |
| | velocity of late atrial filling. Not evaluable in patients with AF. |
| E wave deceleration | measured from mitral valve pulsed wave Doppler represent the |
| time (EDT) | slope of the descending part of E wave. |
| E/A ratio | determines the type of diastolic filling pattern (normal E/A = 1-2 |
| | and EDT = 150-200 ms, abnormal relaxation E/A <1 and |
| | EDT ≥240 ms, pseudonormal E/A = 0.8-1.5; restrictive E/A ≥2 |
| | and EDT <160 ms). Not evaluable in patients with AF. |
| Ea | measured with tissue Doppler method at the lateral and septal |
| | side of the mitral annulus from apical four chamber view is the |
| | early diastolic velocity. The value has been calculated as the |
| | average between Ea lateral and Ea septal. (NR ≥10 cm/s). |
| Aa | measured with tissue Doppler method at the lateral and septal |
| | side of the mitral annulus from apical four chamber view is the |
| | late atrial diastolic velocity. The value has been calculated as |
| | the average between Aa lateral and Aa septal. Not evaluable in |
| | patients with AF |
| E/Ea ratio | this is a derived measure from E and Ea value. This is highly |
| | correlated with left ventricle filling pressure and with prognosis |
| | in patients with HF. |

Left ventricle systolic function parameters

| | |
|---|---|
| Left ventricle ejection | measured with Simpson biplane method according to |
| fraction (LVEF) | international recommendations from apical four chamber view |
| | and apical two chamber view. (NR ≥55%) |
| Sa | measured with tissue Doppler method at the lateral and septal |
| | side of the mitral annulus from apical four chamber view. The |
| | value has been calculated as the average between Sa lateral and |
| | Sa septal. Validation studies demonstrated that Sa correlates |
| | with LVEF (NR ≥6 cm/s). |

Overall cardiac contraction parameters

| | |
|---|---|
| Stroke volume (SV) | is a derived measure obtained with the application of |
| | Bernoulli's formula using the dimension of left ventricle |
| | outflow tract (LVOT) as diameter and LVOT time velocity |
| | integral as velocity. (NR >60 mL/beat) |
| Cardiac output (CO) | is derived by the multiplication of SV × heart rate (HR) (NR: >4 |
| | L/min). |
| Stroke volume index | is a derived parameter obtained by the adjustment of SV by |
| (SVI) | body surface area (BSA) (NR: 33-47 mL/beat/m²). |
| Cardiac index (CI) | is a derived parameter obtained by the adjustment of CO by |
| | body surface area (BSA) (NR: 2.5-4 L/min/m²). |

Right ventricle function parameters

| | |
|---|---|
| Pulmonary arterial | estimated by the sum of the peak velocity at tricuspidal |
| systolic pressure (PASP) | continuous wave Doppler and a fixed value derived from |
| | inferior vena cava diameter and respiratory change. (NR <35 |
| | mmHg). |
| Tricuspid annular | measured from M-mode echocardiography from apical four |
| plane systolic | chamber view. TAPSE correlates with right ventricle ejection |
| excursion (TAPSE) | fraction and its reduction associated with worse prognosis in |
| | HF. (NR >16 mm). |
| Right ventricle Sa | measured with tissue Doppler method at right ventricle free wall |
| | from apical four chamber view. Sa is a derived parameter of |
| | systolic function and correlated with right ventricle ejection |
| | fraction. (NR >10 cm/s). |

Other parameters

| | |
|---|---|
| Mitral regurgitation | evaluated with a visual qualitative assessment ang graded in |
| (MR) | four categories: none, mild, moderate, and severe. |
| Inferior vena cava | measured with M-mode echocardiography from subcostal view |
| diameter (IVC) | at 1-2 cm from the junction with right atrium. This parameter |
| | has been used to estimate systolic pulmonary artery pressure. It |
| | correlated with right atrium pressure indicating the grade of |
| | congestion. Increased IVC diameter is associated with prognosis |
| | in patients with HF (NR: ≤1.5 cm). |

NR = normal range.

In one embodiment, an individual with pre-cardiogenic shock or cardiogenic shock is administered istaroxime, and the SBP AUC₍₀₋₆₎ of the individual increases by at least about 20 mmHg·h within about 6 hours after the start of administration of istaroxime, e.g., 20 mmHg·h, 25 mmHg·h, 30 mmHg·h, 35 mmHg·h, 40 mmHg·h, 45 mmHg·h, 50 mmHg·h, or more within about 6 hours after the start of administration of istaroxime; preferably, the SBP AUC (0-6) increases by at least about 30 mmHg·h; more preferably, it increases by at least about 40 mmHg·h, or at least about 50 mmHg·h. In another embodiment, an individual with pre-cardiogenic shock or cardiogenic shock is administered istaroxime, and the SBP AUC₍₀₋₂₄₎ of the individual increases by at least about 200 mmHg·h within about 24 hours after the start of administration of istaroxime, e.g., 200 mmHg·h, 210 mmHg·h, 220 mmHg·h, 230 mmHg·h, 240 mmHg·h, 250 mmHg·h, 260 mmHg·h, 270 mmHg·h, 280 mmHg·h, 290 mmHg·h, 300 mmHg·h, or more within about 24 hours after the start of administration of istaroxime; preferably, the SBP AUC₍₀₋₂₄₎ increases by at least about 220 mmHg·h; more preferably, it increases by at least about 250 mmHg·h, or at least about 280 mmHg·h. When compared to individuals with pre-cardiogenic shock or cardiogenic shock who are not administered istaroxime, an individual with pre-cardiogenic shock or cardiogenic shock who is administered istaroxime by infusion will have a SBP AUC₍₀₋₆₎ that is at least about 30% greater, e.g., 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70% or more than the mean SBP AUC₍₀₋₆₎ for those individuals who are not administered istaroxime; preferably, the individual will have a SBP AUC₍₀₋₆₎ that is at least about 40% greater than the mean SBP AUC₍₀₋₆₎ for those individuals who are not administered istaroxime; more preferably, the individual will have a SBP AUC₍₀₋₆₎ that is at least about 50% greater than the mean SBP AUC₍₀₋₆₎ for those individuals who are not administered istaroxime, or at least about 60% greater than the mean AUC₍₀₋₆₎ for those individuals who are not administered istaroxime, or at least about 70% greater than the mean AUC₍₀₋₆₎ for those individuals who are not administered istaroxime. In another embodiment, an individual with pre-cardiogenic shock or cardiogenic shock who is administered istaroxime by infusion will have a SBP AUC₍₀₋₂₄₎ that is at least about 10% greater, e.g., 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, or more than the mean SBP AUC₍₀₋₂₄₎ for those individuals who are not administered istaroxime; preferably, the individual will have a SBP AUC₍₀₋₂₄₎ that is at least about 20% greater than the mean SBP AUC₍₀₋₂₄₎ for those individuals who are not administered istaroxime; more preferably, the individual will have a SBP AUC₍₀₋₆₎ that is at least about 30% greater than the mean SBP AUC₍₀₋₂₄₎ for those individuals who are not administered istaroxime, or at least about 40% greater than the mean AUC₍₀₋₂₄₎ for those individuals who are not administered istaroxime.

In another embodiment, the change in SBP increases by at least about 1 mmHg within about 1 hour after the start of infusion, or by at least about 3 mmHg within about 3 hours after the start of infusion, or by at least about 8 mmHg without about 6 hours after the start of infusion, or by at least about 10 mmHg without about 24 hours after the start of infusion. For instance, in one particular embodiment, the adjusted increase in SBP at 6 hours after infusion is at least about 8 mmHg, e.g., 8 mmHg, 9 mmHg, 10 mmHg, 11 mmHg, 12 mmHg, 13 mmHg, 14 mmHg, or 15 mmHg; preferably the adjusted increase in SBP at 6 hours after infusion is at least about 10 mmHg. In another embodiment, the adjusted increase in SBP at 24 hours after infusion is at least about 10 mmHg, e.g., 10 mmHg, 11 mmHg, 12 mmHg, 13 mmHg, 14 mmHg, or 15 mmHg, 16 mmHg, 17 mmHg, 18 mmHg, 19 mmHg, or 20 mmHg; preferably the adjusted increase in SBP at 24 hours after infusion is at least about 12 mmHg; more preferably, it is at least about 15 mmHg.

In addition to the increased SBP, individuals with pre-cardiogenic shock or cardiogenic shock administered istaroxime will exhibit an improvement in one or more additional parameters of cardiac function, including, but not limited to, increased cardiac index (CI), decreased left atrial area, increased stroke volume, decreased left ventricular end-systolic volume, and decreased left ventricular end-diastolic volume. Interestingly, istaroxime administration improved CI by increasing the stroke rate, but not by increasing the heart rate. As such, istaroxime increases cardiac output in these individuals without additional strain of increased heart rate on the heart.

In another embodiment, blood is drawn from the individual, e.g., during prescreening, at baseline and/or at various timepoints post-infusion, e.g., 12, 24, 48, 72, 96 hours, 5 days, 10 days, 30 days after the start of istaroxime administration. This blood can be used for various laboratory testing to assess the individual's body chemistry, hematology, heart protein levels, and the pharmacokinetics of the istaroxime infusion. Exemplary chemistry includes, but is not limited to, electrolytes, liver function tests, lipids, creatinine (and estimated glomerular filtration rate (eGFR)), urea, blood urea nitrogen (BUN), glucose, albumin, and protein. Exemplary hematology includes, but is not limited to, complete blood count with differential. Suitable heart proteins include cardiac troponin I (cTNI), cardiac troponin T (cTNT), N-terminal pro b-type natriuretic peptide (NT-proBNP), and brain natriuretic peptide (BNP). The levels of each most of these proteins provide information to the practitioner about heart function and the measurement and/or interpretation of these levels is well within the purview of the skilled artisan. For example, in one embodiment, the level of NT-proBNP is decreased as compared to baseline within about 6 hour of infusion, or within about 24 hours of administration. The levels of some of parameters are not a direct measure of heart function, but rather are used to assess kidney function. While many inotropic or vasopressor drugs have been shown to have a detrimental effect on renal function, individuals administered istaroxime exhibit improved cardiac function without the deleterious impact on renal function. For instance, in one embodiment, in an individual with pre-cardiogenic shock or cardiogenic shock administered istaroxime for a period of 24 hours, the level of eGFR, which is a measure of kidney function, is increased as compared to baseline within about 6 hours of infusion, or within about 24 hours of infusion.

Yet other parameters of heart function include chest congestion, which can be measured by use of a chest x-ray, lung ultrasound, or other art-standard equipment, wherein an improvement in the level of chest congestion following istaroxime infusion indicates treatment or prevention of pre-cardiogenic shock and/or cardiogenic shock. Yet others include heart rate, mean arterial pressure (MAP), dyspnea or labored breathing, need for administration of intravenous vasopressors, other inotropes, and/or mechanical cardiac or renal support, symptoms of worsening heart failure, incident of adverse events (AEs) or serious adverse events (SAEs), need for admission to intensive care unit, discharge from the intensive care unit, and death. In some embodiments, MAP is increased as compared to baseline within about 24 hours of infusion.

### Pharmaceutical Compositions

Pharmaceutical compositions and formulations for intravenous infusion comprising istaroxime or a pharmaceutically acceptable salt, solvate, or hydrate thereof in admixture with at least one conventional pharmaceutically acceptable carrier and/or vehicle and/or excipient are commonly known in the art.

The pharmaceutical compositions and formulations for intravenous infusion can be formulated in any way and can be administered in a variety of unit dosage forms depending upon the condition or disease and the degree of illness, the general medical condition of each patient, the resulting preferred method of administration and the like. Details on techniques for formulation and administration are well described in the scientific and patent literature [see, e.g., the latest edition of Remington's Pharmaceutical Sciences, Mack Publishing Co, Easton PA (“Remington's”)].

The formulations may conveniently be presented in unit dosage form and may be prepared by any method known in the art of pharmacy. The amount of active ingredient which can be combined with a carrier or vehicle material to produce a single dosage form will vary depending upon the subject being treated and the particular mode of administration. The amount of active ingredient that can be combined with a carrier material to produce a single dosage form will generally be the amount of the compound which produces a therapeutic effect.

Pharmaceutical formulations as provided herein can be prepared according to any method known to the art for the manufacture of pharmaceuticals. Such formulations can contain additional agents, such as preserving or stabilizing agents. A formulation can be admixtured with nontoxic pharmaceutically acceptable carriers or excipients which are suitable for manufacture. Formulations may comprise one or more diluents, emulsifiers, preservatives, buffers, excipients, etc. and may be provided in such forms as liquids, powders, emulsions, lyophilized powders, etc.

Aqueous suspensions can contain an active agent (e.g., a composition used to practice the uses and methods as provided herein) in an admixture with excipients suitable for the manufacture of aqueous suspensions. Such excipients include a suspending agent, such as sodium carboxymethylcellulose, methylcellulose, hydroxypropyl-methylcellulose, sodium alginate, polyvinylpyrrolidone, gum tragacanth and gum acacia, and dispersing or wetting agents, such as a naturally-occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate. Formulations can be adjusted for osmolarity.

According to the present invention, istaroxime is given by intravenous (IV) administration. These formulations can comprise a solution of active agent dissolved in a pharmaceutically acceptable carrier. Acceptable vehicles and solvents that can be employed are water, dextrose in water, and Ringer's solution, an isotonic sodium chloride. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of active agent in these formulations can vary widely, and will be selected primarily based on fluid volumes, viscosities, body weight, and the like, in accordance with the particular mode of administration selected and the patient's needs. The administration is by bolus or continuous infusion (e.g., substantially uninterrupted introduction into a blood vessel for a specified period of time).

Istaroxime as provided herein can be lyophilized. Provided herein is a stable lyophilized formulation comprising a composition as provided herein, which can be made by lyophilizing a solution comprising a pharmaceutical as provided herein and a bulking agent, e.g., mannitol, trehalose, raffinose, lactose, and sucrose or mixtures thereof. There are many other conventional lyophilizing agents. Among the sugars, lactose is the most common. Also used are citric acid, sodium carbonate, EDTA, Benzyl alcohol, glycine, sodium chloride, etc. [see, for example, Journal of Excipients and Food Chemistry Vol. 1, Issue 1 (2010) pp 41-54; U.S. patent app. no. 20040028670]. In a preferred embodiment, istaroxime can be prepared as a lyophilized powder for injection according to the teaching of CN103315968.

According to the present invention, istaroxime as provided herein can be administered for prophylactic and/or therapeutic treatments. In therapeutic applications, compositions are administered to a subject already suffering from a condition, or disease in an amount sufficient to treat, prevent, cure, alleviate or partially arrest the clinical manifestations of the condition, or disease and its complications (i.e., a “therapeutically effective amount”). For example, in alternative embodiments, pharmaceutical compositions as provided herein are administered in an amount sufficient to treat, prevent or ameliorate in an individual in need thereof. The amount of pharmaceutical composition adequate to accomplish this is defined as a “therapeutically effective dose.” The dosage schedule and amounts effective for this use, i.e., the “dosing regimen,” will depend upon a variety of factors, including the stage of the disease or condition, the severity of the disease or condition, the general state of the patient's health, the patient's physical status, age and the like. In calculating the dosage regimen for a patient, the mode of administration also is taken into consideration.

The dosage regimen also takes into consideration pharmacokinetics parameters well known in the art, i.e., the active agents' bioavailability, metabolism, clearance, and the like [see, e.g., Hidalgo-Aragones, 1996, Steroid Biochem. Mol. Biol. 58:611-617; Groning, 1996, Pharmazie 51:337-341; Fotherby, 1996, Contraception 54:59-69; Johnson, 1995, J. Pharm. Sci. 84:1144-1146; Rohatagi, 1995, Pharmazie 50:610-613; Brophy, 1983, Eur. J. Clin. Pharmacol. 24:103-108; the latest Remington's, supra]. The state of the art allows the clinician to determine the dosage regimen for each individual patient, active agent and disease or condition treated. Guidelines provided for similar compositions used as pharmaceuticals can be used as guidance to determine the dosage regimen, i.e., dose schedule and dosage levels, administered practicing the methods as provided herein are correct and appropriate.

Single or multiple administrations of formulations can be given depending on the dosage and frequency as required by the pre-cardiogenic shock or cardiogenic shock symptoms of patient. The formulations should provide a sufficient quantity of active agent to effectively treat or prevent or ameliorate a conditions, diseases or symptoms as described herein. A correct treatment of pre-cardiogenic shock or cardiogenic shock, by selectively normalizing a depressed biochemical activity underlying the symptoms of subset of patients, may be expected to selectively improve the symptoms and to reduce the incidence of unwanted side effects produced by the available drugs either during hospital staying or after discharge. The term prevention is applicable when the continuous monitoring of the pulmonary pressure is possible with the appropriate chronic implantable devices that furnish and estimation of SBP, SBP AUC (e.g., to 6 hours or to 24 hours) or other parameter of heart function as discussed elsewhere herein.

In one embodiment, an effective amount of istaroxime or equivalent of a pharmaceutically acceptable salt, solvate, or hydrate thereof, is administered to an individual with pre-cardiogenic or cardiogenic shock symptoms at a dosing schedule, e.g., from about 0.1 μg/kg/min to about 3.0 μg/kg/min, e.g., 0.1 μg/kg/min, 0.15 μg/kg/min, 0.2 μg/kg/min, 0.25 μg/kg/min, 0.3 μg/kg/min, 0.35 μg/kg/min, 0.4 μg/kg/min, 0.45 μg/kg/min, 0.5 g/kg/min, 0.55 μg/kg/min, 0.6 μg/kg/min, 0.7 μg/kg/min, 0.75 μg/kg/min, 0.8 μg/kg/min, 0.85 μg/kg/min, 0.9 μg/kg/min, 0.95 μg/kg/min, 1.0 μg/kg/min, 1.1 μg/kg/min, 1.2 μg/kg/min, 1.3 μg/kg/min, 1.4 μg/kg/min, 1.5 μg/kg/min, 1.6 μg/kg/min, 1.7 μg/kg/min, 1.8 μg/kg/min, 1.9 μg/kg/min, 2.0 μg/kg/min, 2.1 μg/kg/min, 2.2 μg/kg/min, 2.3 μg/kg/min, 2.4 μg/kg/min, 2.5 μg/kg/min, 2.6 μg/kg/min, 2.7 g/kg/min, 2.8 μg/kg/min, 2.9 μg/kg/min, or 3.0 μg/kg/min. For instance, in some embodiments, istaroxime is administered by infusion at an effective dose from about 0.2 μg/kg/min to about 2.0 g/kg/min, or from about 0.5 g/kg/min to about 1.5 μg/kg/min, or from about 1.0 μg/kg/min to about 1.5 g/kg/min. In one particular embodiment, the istaroxime is administered by intravenous infusion at an effective dose of about 1.5 μg/kg/min. Alternatively, the effective dose of istaroxime can be initiated at about 1.0 μg/kg/min for about 1 to about 2 hours, which is then increased to 1.5 μg/kg/min for the remaining infusion time unless the individual exhibits nausea or other signs of poor or insufficient drug tolerance. In another embodiment, the effective dose of istaroxime can be initiated at about 1.5 μg/kg/min for about 1 to about 2 hours, which is then increased to 1.0 μg/kg/min for the remaining infusion time.

In one exemplary embodiment, a pharmaceutical composition comprising istaroxime in lyophilized power form is provided. In another embodiment, the pharmaceutical composition comprises istaroxime and a bulking agent (e.g., lactose) in lyophilized powder form. For instance, the composition may be a lyophilized powder comprising about 0.1 to about 0.3 wt % istaroxime and about 0.7 to about 0.9 wt % lactose (e.g., 10 mg istaroxime and 50 mg lactose). The lyophilized powder can be reconstituted with any pharmaceutically acceptable carrier, such as saline with 0.9% NaCl) prior to intravenous infusion.

### Methods of Treatment

Also provided herein are pharmaceutical compositions for use as a medicament in the treatment of pre-cardiogenic shock or cardiogenic shock as well as methods of treating an individual with pre-cardiogenic shock or cardiogenic shock. In one embodiment, the individual exhibits symptoms of, or has been diagnosed with pre-cardiogenic shock SCAI Stage B. In a preferred embodiment, the individual exhibits symptoms of, or has been diagnosed with SCAI Stage B pre-cardiogenic shock caused by ADHF. In a more preferred embodiment, the individual exhibits symptoms of, or has been diagnosed with SCAI Stage B pre-cardiogenic shock caused by ADHF without evidence for an acute coronary syndrome (e.g., myocardial infarction). For instance, the individual have dyspnea at rest or with minimal exertion, chest congestion with BNP equal to or greater than about 400 μg/mL or NT-proBNP equal to or greater than about 1,400 μg/mL. Preferably, the individual will exhibit persistent hypotension characterized by an SBP for between about 70 mmHg and about 100 mmHg or, more preferably between about 75 mmHg and about 90 mmHg for at least 2 hours prior to screening or initiation of treatment. Further, the SBP in this individual does not decrease by greater than about 7 mmHg before initiation of treatment. The individual may also have a heart rate in the range from about 65 bpm to about 160 bpm or, preferably about 75 bpm to about 150 bpm or, if the individual is on a beta-blocker, the heart rate might be from about 60 bpm to about 150 bpm. The individual may also have a history of LVEF less than or greater to about 40%. In another embodiment, the individual has a heart rate of less than about 90 mmHg without evidence of end-organ hypoperfusion. Alternatively, in some embodiments, the individual has a heart rate of less than about 90 mmHg with evidence of end-organ hypoperfusion. In another embodiment, the individual exhibits symptoms or, or has been diagnosed with SCAI Stage C cardiogenic shock. However, in other embodiments, the treatment is directed to treating pre-cardiogenic shock and the individual will not exhibit symptoms of, or be diagnosed with SCAI Stage C cardiogenic shock or have evidence of end-organ hypoperfusion. While the individual can be a non-human animal, in a preferred embodiment, the individual is a human patient, such as a human patient exhibiting pre-cardiogenic shock symptoms.

In some cases, a measuring step is first carried out to determine the baseline heart function of the individual. For instance, an individual with heart failure may present with persistent hypotension, dyspnea, and reduced LVEF. The measuring step may include measuring one or more parameters of heart function or heart function impairment, such as, but not limited to, decreased SBP or SBP AUC, reduced left ventricular end-diastolic/systolic volume and function (LVEF), or increased E/Ea or E/A ratios, reduced Ea ratio decreased stroke volume, elevated heart rate, and others. As one having ordinary skill in the art will appreciate, any suitable measuring technique available in the art at the time of the measuring step is suitable for use herein, and it is well within the purview of such skilled artisan to select an appropriate measuring technique corresponding to the parameter of interest. A non-limiting list of suitable measuring equipment/techniques includes echocardiogram, cardiac catheterization, nuclear stress test, CAT scan, radionuclide ventriculography scan, stethoscope, sphygmomanometer, pulmonary capillary wedge pressure (PCWP), and the like. For instance, the SBP can be measured by sphygmomanometer or arterial line attached to a pressure transducer. In another embodiment, the one or more parameters of heart function are measured and selected from the group consisting of heart rate (HR), blood pressure (BP), diastolic relaxation, systolic contraction, dyspnea, chest congestion, diastolic blood pressure (DBP), systolic blood pressure (SBP), systolic blood pressure area under the curve (SBP AUC), creatine clearance, deceleration slope, mitral inflow velocity, mean arterial pressure, brain natriuretic peptide levels (BNP), NT-pro-BNP levels, troponin levels, venous lactate levels, echocardiographic measurements, left ventricle end diastolic diameter (EDD), left ventricle end systolic diameter (ESD), left ventricle end diastolic volume (EDV), left ventricle end systolic volume (ESV), left atrium diameter (LAD), left atrium area (LAA), left atrium volume (LAV), E wave, A wave, E wave deceleration time (EDT), E/A ratio, Ea, Aa, E/Ea ratio, left ventricle ejection fraction (LVEF), Sa, stroke volume (SV), cardiac output (CO), stroke volume index (SVI), cardiac index (CI), pulmonary arterial systolic pressure (PASP), tricuspid annular plane systolic excursion (TAPSE), right ventricle Sa, mitral regurgitation (MR), and inferior vena cava diameter (IVC).

It may be desirable to develop a clinical study group to examine a cohort of individuals exhibiting heart function parameters within a selected range of values. For such a study, as one having ordinary skill in the art will appreciate, there are certain inclusion criteria and exclusion criteria for determine whether or not an individual will be selected for participating in the study. In one embodiment, an individual is selected for treatment having one or more inclusion criteria. In a preferred embodiment, the inclusion criteria includes one or more of clinical presentation with SCAI Stage B pre-cardiogenic shock with ADHF and no evidence of acute coronary syndrome. In another embodiment, additional inclusion criteria include males and females that are 18 to 85 years of age, dyspnea, chest congestion with BNP equal to or greater than about 400 μg/mL or NT-proBNP equal to or greater than about 1,400 μg/mL, LVEF of less than or equal to 40%, persisting hypotension defined as an SBP of less than about 90 mmHg, and/or heart rate of about 75 bpm to about 150 bpm.

The methods disclosed herein also include administering to the individual a therapeutically effective amount of istaroxime. In a preferred embodiment, the istaroxime is in a pharmaceutical composition, such as any of the combinations discussed above. The istaroxime is administered in a therapeutically effective dose. For instance, the istaroxime can be administered to the individual via intravenous infusion with a dose in the range from about 0.25 μg/kg/min to about 3.0 μg/kg/min, e.g., 0.25 μg/kg/min, 0.3 μg/kg/min, 0.35 μg/kg/min, 0.4 μg/kg/min, 0.45 μg/kg/min, 0.5 μg/kg/min, 0.55 μg/kg/min, 0.6 μg/kg/min, 0.65 μg/kg/min, 0.7 μg/kg/min, 0.75 μg/kg/min, 0.8 μg/kg/min, 0.85 μg/kg/min, 0.9 μg/kg/min, 0.95 μg/kg/min, 1.0 μg/kg/min, 1.1 μg/kg/min, 1.2 μg/kg/min, 1.3 μg/kg/min, 1.4 μg/kg/min, 1.5 μg/kg/min, 1.6 μg/kg/min, 1.7 μg/kg/min, 1.8 μg/kg/min, 1.9 μg/kg/min, 2.0 μg/kg/min, 2.1 g/kg/min, 2.2 μg/kg/min, 2.3 μg/kg/min, 2.4 μg/kg/min, 2.5 g/kg/min, 2.6, 2.7 μg/kg/min, 2.8 μg/kg/min, 2.9 μg/kg/min, or 3.0 μg/kg/min. In a preferred embodiment, the dose of istaroxime administered to the individual is in the range from about 0.5 μg/kg/min to about 3.0 μg/kg/min. In a more preferred embodiment, the dose of istaroxime administered to the individual is in the range from about 1.0 μg/kg/min to about 2.0 μg/kg/min, or about 1.0 μg/kg/min to about 1.5 μg/kg/min, or about 1.0 μg/kg/min, or about 1.5 μg/kg/min. In one particular embodiment, the dose of istaroxime administered to the individual is about 1.0 μg/kg/min.

The time period for the infusion may be at least about 1 hour to about 48 hours, or more, e.g., 1 h, 2 h, 3 h, 4 h, 5 h, 6 h, 7 h, 8 h, 9 h, 10 h, 11 h, 12 h, 13 h, 14 h, 15 h, 16 h, 17 h, 18 h, 19 h, 20 h, 21 h, 22 h, 23 h, 24 h, 25 h, 26 h, 27 h, 28 h, 29 h, 30 h, 31 h, 32 h, 33 h, 34 h, 35 h, 36 h, 37 h, 38 h, 39 h, 40 h, 41 h, 42 h, 43 h, 44 h, 45 h, 46 h, 47 h, or 48 h. Preferably, the infusion is carried out from about 3 hours to about 24 hours, or from about 6 hours to about 24 hours, or from about 12 hours to about 24 hours. In one particular embodiment, the individual is administered istaroxime by intravenous infusion for about 24 hours. In other embodiments, the time period for infusion may be greater than 24 hours, e.g., 25 h, 26 h, 27 h, 28 h, 29 h, 30 h, 31 h, 32 h, 33 h, 34 h, 35 h, 36 h, 37 h, 38 h, 39 h, 40 h, 41 h, 42 h, 43 h, 44 h, 45 h, 46 h, 47 h, 48 h, 54 h, 60 h, 66 h, 72 h, 78 h, 84 h, 90 h, 96 h, 5 days, 6 days, 7 days, 8 days, 9 days, 10 days, 11 days 12 days, 13 days, 14 days, or longer. For instance, istaroxime infusion administered by a pump can be carried out for much longer duration.

In another embodiment, the individual is administered a lower dose of istaroxime for an initial period of time to determine whether that individual exhibits signs for poor drug tolerance, such as nausea, infusion site pain, etc. For instance, the individual can be administered istaroxime by intravenous infusion at a dose of about 1.0 μg/kg/min for an initial 1-2 hours, which dose can be increased to 1.5 μg/kg/min for the remaining infusion time (e.g., 24 hours) if the individual does not exhibit signs of drug intolerance. Similarly, the individual can be administered a higher dose of istaroxime initially, which can be lowered if the individual exhibits signs of drug intolerance. For instance, the individual can be administered istaroxime by intravenous infusion at a dose of about 1.5 g/kg/min for an initial 1-2 hours, which dose can be decreased to 1.0 μg/kg/min for the remaining infusion time (e.g., 24 hours) if the individual does exhibits signs of drug intolerance, such as nausea. In some embodiments, the individual can be co-administered an antiemetic (e.g., ondansetron) to alleviate nausea as an alternative to adjusting the infusion dose of istaroxime.

Once the individual is administered istaroxime, the method can include one or more measuring steps carried out periodically during the treatment and/or for a period of time post-treatment. The measuring steps may include measuring one or more parameters of heart function as described above prior to beginning infusion, at the initiation of infusion administration, and/or at one or more time points during and after infusion, e.g., 15 m, 30 m, 45 m, 1 h, 1.25 h, 1.5 h, 1.75 h, 2 h, 3 h, 4 h, 5 h, 6 h, 7 h, 8 h, 9 h, 10 h, 11 h, 12 h, 13 h, 14 h, 15 h, 16 h, 17 h, 18 h, 19 h, 20 h, 21 h, 22 h, 23 h, 24 h, 48 h, 72 h, 96 h, 5 days, 6 days, 7 days, 8 days, 9 days, 10 days, 14 days, 21 days, 28 days, 30 days, or more following the start of infusion. For instance, for an individual administered istaroxime by intravenous infusion for up to 24 hours, one or more parameters of heart function can be measured before, during, and 12 h, 24 h, 48, h, 72 h, 96 h, and 30 days following initiation of infusion. As one having ordinary skill in the art would appreciate, the one or more parameters of heart function indicate the efficacy of istaroxime treatment. In some embodiments, an increase in SPB by at least about 8 mmHg, e.g., 8 mmHg, 9 mmHg, 10 mmHg, 11 mmHg, 12 mmHg, 13 mmHg, 14 mmHg, or 15 mmHg after 6 hours of istaroxime administration indicates treatment of pre-cardiogenic shock or prevention of cardiogenic shock; or an increase in SPB by at least about 10 mmHg, e.g., 10 mmHg, 11 mmHg, 12 mmHg, 13 mmHg, 14 mmHg, or 15 mmHg, 16 mmHg, 17 mmHg, 18 mmHg, 19 mmHg, or 20 mmHg after 24 hours of istaroxime administration indicates treatment of pre-cardiogenic shock or prevention of cardiogenic shock. In other embodiments, an increase in SPB AUC (0-6) from baseline of at least about 20 mmHg·h, e.g., 20 mmHg·h, 25 mmHg·h, 30 mmHg·h, 35 mmHg·h, 40 mmHg·h, 45 mmHg·h, 50 mmHg·h, or more within about 6 hours after the start of administration of istaroxime indicates treatment of pre-cardiogenic shock or prevention of cardiogenic shock. Further, an increase in SBP AUC₍₀₋₂₄₎ from baseline of at least about 200 mmHg·h, e.g., 200 mmHg·h, 210 mmHg·h, 220 mmHg·h, 230 mmHg·h, 240 mmHg·h, 250 mmHg·h, 260 mmHg·h, 270 mmHg·h, 280 mmHg·h, 290 mmHg·h, 300 mmHg·h, or more within about 24 hours after the start of administration of istaroxime indicates treatment of pre-cardiogenic shock or prevention of cardiogenic shock. In some embodiments, the improvement of SBP is accompanied by one or more additional indicators of heart function improvement, such as, but not limited to, increased CI, decreased left ventricular dimensions (e.g., decreased left ventricular end-systolic volume and/or decreased left ventricular end-diastolic volume), and/or decreased left atrial dimensions (e.g., decreased left atrial area). In other embodiments, a decrease in the level of NT-proBNP or an increase in the level of eGFP indicates treatment of pre-cardiogenic shock or prevention of cardiogenic shock.

In alternative embodiments, in evaluating the efficacy of a treatment, a treatment regimen or a particular dosage, or to determine if a treatment versus a maintenance dosage should be given, individuals, e.g., patients affected by pre-cardiogenic shock or cardiogenic shock, are subject to regular periodic screening for the presence and extent of organ and tissue involvement or damage, e.g., heart (ventricle dilatation, third heart sound cardiac hypertrophy), fatigue, tiredness, reduced exercise tolerance, increased time to recover after exercise, kidney (renal insufficiency, oliguria), lung (orthopnea, paroxysmal nocturnal dyspnea, tachypnea), ankle swelling, elevated jugular venous pressure. A thorough physical examination should be done at a time interval chosen by those experts in the treatment of a cardiovascular disease, in particular, ADHF with pre-cardiogenic shock or cardiogenic shock which would concentrate on cardiac, pulmonary and peripheral circulation functions. Accordingly, in alternative embodiments, therapy with istaroxime or an equivalent of a pharmaceutically acceptable salt, solvate or hydrate thereof as disclosed herein, is instituted as early as possible, preferably in emergency, to prevent the rapid evolution of symptoms (e.g., progression from pre-cardiogenic shock to overt cardiogenic shock) and continued after patient's discharge for years, preferably during the whole life of the patient or at least a period consistent with the way other drugs are used in heart failure.

According to the present invention, uses and methods as provided herein can further comprise co-administration with other drugs or pharmaceuticals. In fact, the present invention selectively corrects a depressed cardiac biochemical function (namely the SERCA2a activity). This certainly contributes to relieving the existing pre-cardiogenic shock or cardiogenic shock and ADHF clinical symptoms, with less unwanted side effects than those of the available therapies (just because the selectivity mentioned above). However, as pre-cardiogenic shock and cardiogenic shock are complex clinical syndromes the present invention is potentially associable to existing and future drug classes and/or specific drugs such as: a) drug classes such as, ACE inhibitors, angiotensin receptor blockers (ARBs), diuretics, Ca channel blockers, beta blockers, digitalis, NO donors, vasodilators, SERCA2a stimulators, neprilysin (NEP) inhibitors, myosin filament activators, recombinant relaxin-2 mediators, recombinant NP protein, activators of the soluble Guanylate Cyclase (sGC), beta-arrestin ligand of Angiotensin II receptor; b) specific drugs: hydrochlorothyzide, furosemide, verapamil, diltiazem, carvedilol, metoprolol, hydralazine, eplerenone, spironolactone, lisinopril, ramipril, nitroglycerin, nitrates, digoxin, valsartan, olmesartan, telmisartan, candesartan, losartan, entresto, omecamtiv, sacubitril, serelaxin, ularitide, levosimendan, cinaciguat. Subjects suffering from pre-cardiogenic shock or cardiogenic shock treated with the above drugs and undergoing regular clinical monitoring, for example having their pulmonary blood pressure continuously monitored with implanted probes, can be guarded in order to predict episode of ADHF or cardiogenic shock that may be prevented by the infusion of istaroxime according to the present invention.

Istaroxime as disclosed in the present invention, as used a therapeutic agent for treating pre-cardiogenic shock or cardiogenic shock, can be combined or co-adminstered with other therapeutic agents used in the treatment of the same disease and/or the underlying HF. Exemplary other therapeutic agents are diuretics, for example furosemide, bumetanide, and torasemide. Metolazone, an aldosterone antagonist, such as spironolactone or eplerenone; thiazide diuretics, such as Hydrochlorothiazide, metolazone, and chlorthalidone. Other agents are ACE inhibitors, for example Lisinopril and Ramipril. Also ARBs, such as valsartan, candesartan and losartan can be taken into consideration. Angiotensin receptor/neprilysin inhibitor (ARNI), sacubitril for example, are comprised. Other agents can be selected from Beta-blockers, such as carvedilol and metoprolol for example, or Vasodilators, for example Hydralazine, optionally combined with isosorbide dinitrate, Nitrates, as nitroglycerin, amlodipine and felodipine; non-dihydropyridines such as diltiazem or verapamil. The compounds of the present invention can also be combined with Digoxin, if needed. Other drugs, as Ivabradine and other Anticoagulant may be considered.

The compounds of the present invention can be combined with other therapeutic agents, in particular agents useful for treating cardiovascular diseases, more in particular in the combination therapy of pre-cardiogenic shock or cardiogenic shock with ADHF. The combined active ingredients can be administered according to different protocols, decided by the medical doctor. According to an embodiment of the present invention, combination therapy can be carried out by administering istaroxime both at the same time or at different time of the further therapeutically active ingredient or ingredients. In case of concomitant administration, the compound of the present invention and the further active ingredient or ingredients can be each formulated in a respective pharmaceutical composition or in the same unitary dosage form. In the former case, the present invention provides a kit, in particular for the treatment of pre-cardiogenic shock or cardiogenic shock, comprising separate pharmaceutical compositions containing the compound of the present invention and the further active ingredient or ingredients, respectively. In another embodiment, the present invention provides a pharmaceutical unit dosage form kit, in particular for the treatment of pre-cardiogenic shock or cardiogenic shock, comprising compound of the present invention and the further active ingredient or ingredients in the same unit dosage form. Combination therapy according to the present invention provides advantageous treatment of pre-cardiogenic shock or cardiogenic shock due to the inotropic-lusitropic effect of istaroxime herein disclosed in addition to or synergically combined with the well-known therapeutic effect of the additional active agents herein disclosed.

Also provided are nanoparticles, nanolipoparticles, vesicles and liposomal membranes comprising compounds used to practice the uses and methods as provided herein, e.g., to deliver pharmaceutically active compounds and compositions as provided herein: istaroxime or an equivalent of a pharmaceutically acceptable salt, solvate or hydrate thereof, optionally combined with a further therapeutically active agent as disclosed above to a subject in need thereof. In alternative embodiments, these compositions are designed to target specific molecules, including biologic molecules, such as polypeptides, including cell surface polypeptides, e.g., for targeting a desired cell type, e.g., a myocyte or heart cell, an endothelial cell, and the like. A slow release of Istaroxime may provide a sufficient compound to selectively increase the plasma levels of the metabolite leaving the plasma levels of Istaroxime within very low ranges.

Provided are multilayered liposomes comprising compounds used to practice methods as provided herein, e.g., as described in U.S. application No. 20070082042. The multilayered liposomes can be prepared using a mixture of oil-phase components comprising squalane, sterols, ceramides, neutral lipids or oils, fatty acids and lecithins, to about 200 to 5000 nm in particle size, to entrap a composition used to practice uses and methods as provided herein.

Liposomes can be made using any method, e.g., as described in U.S. Pat. No. 4,534,899; or U.S. application No. 20070042031, including method of producing a liposome by encapsulating an active agent according to the present invention (or a combination of active agents), the method comprising providing an aqueous solution in a first reservoir; providing an organic lipid solution in a second reservoir, and then mixing the aqueous solution with the organic lipid solution in a first mixing region to produce a liposome solution, where the organic lipid solution mixes with the aqueous solution to substantially instantaneously produce a liposome encapsulating the active agent; and immediately then mixing the liposome solution with a buffer solution to produce a diluted liposome solution.

In one embodiment, liposome compositions used to practice uses and methods as provided herein comprise a substituted ammonium and/or polyanions, e.g., for targeting delivery of istaroxime or an equivalent of a pharmaceutically acceptable salt, solvate or hydrate thereof used to practice methods as provided herein to a desired cell type, as described, e.g., in U.S. application No. 20070110798.

Provided are nanoparticles comprising compounds according to the present invention used to practice uses and methods as provided herein in the form of active agent-containing nanoparticles (e.g., a secondary nanoparticle), as described, e.g., in U.S. application No. 20070077286. In one embodiment, provided are nanoparticles comprising a fat-soluble active agent used to practice a use and method as provided herein or a fat-solubilized water-soluble active agent to act with a bivalent or trivalent metal salt.

In one embodiment, solid lipid suspensions can be used to formulate and to deliver compositions used to practice uses and methods as provided herein to mammalian cells in vivo, in vitro or ex vivo, as described, e.g., in U.S. application No. 20050136121.

The compositions and formulations used to practice the uses and methods as provided herein can be delivered by the use of liposomes or nanoliposomes. By using liposomes, particularly where the liposome surface carries ligands specific for target cells, or are otherwise preferentially directed to a specific organ, one can focus the delivery of the active agent into target cells in vivo [see, e.g., U.S. Pat. Nos. 6,063,400; 6,007,839; Al-Muhammed, 1996, Microencapsul. 13:293-306; Chonn, 1995, Curr. Opin. Biotechnol. 6:698-708; Ostro, 1989, Am. J. Hosp. Pharm. 46:1576-1587.] A liposome formulation of istaroxime as disclosed in Eur J Pharm Biopharm. 2011; 79 (2): 285-93 is also provided in the present invention.

### Delivery Vehicles

In alternative embodiments, any delivery vehicle can be used to practice the uses and methods as provided herein, e.g., to deliver the compounds provided herein to a subject in need thereof. For example, delivery vehicles comprising polycations, cationic polymers and/or cationic peptides, such as polyethyleneimine derivatives, can be used e.g. as described, e.g., in U.S. application No. 20060083737.

In one embodiment, a dried polypeptide-surfactant complex is used to formulate a composition used to practice a use and method as provided herein, e.g., as described in U.S. application No. 20040151766.

In one embodiment, a composition used to practice uses and methods as provided herein can be applied to cells using vehicles with cell membrane-permeant peptide conjugates, e.g., as described in U.S. Pat. Nos. 7,306,783; 6,589,503. In one aspect, the composition to be delivered is conjugated to a cell membrane-permeant peptide. In one embodiment, the composition to be delivered and/or the delivery vehicle are conjugated to a transport-mediating peptide, e.g., as described in U.S. Pat. No. 5,846,743, describing transport-mediating peptides that are highly basic and bind to poly-phosphoinositides.

In one embodiment, electro-permeabilization is used as a primary or adjunctive means to deliver the composition to a cell, e.g., using any electroporation system as described e.g. in U.S. Pat. Nos. 7,109,034; 6,261,815; 5,874,268.

The following examples further illustrate the present invention.

### Example 1—Treatment with 24 Hour Istaroxime Infusion in Patients Hospitalized for Pre-Cardiogenic Shock: A Randomized, Double-Blind, Placebo-Controlled Parallel Group Study

To assess the safety and efficacy of istaroxime in treating patients with pre-cardiogenic shock, sixty patients with AHF and exhibiting pre-cardiogenic shock (pre-CS) without acute myocardial infarction were randomized to istaroxime 1.0-1.5 mg/kg/min or placebo for 24 hours. Pre-CS was defined as defined as systolic blood pressure (SBP)<90 mmHg without hypoperfusion, venous lactate >2 mmol/L and/or mechanical or inotropic support. The inclusion criteria included AHF-related SCAI stage B pre-CS, 18-85 years of age, an ongoing hospitalization for AHF, left ventricular ejection fraction <40%, persistent hypotension (SBP between 75 and 90 mmHg), heart rate of 75-150 bpm and no need at time of screening or planned use for 6 h thereafter of mechanical support or intravenous therapy to increase BR According to the stage B SCAI classification, patients with clinical signs of peripheral hypoperfusion, venous lactate >2 mmol/L and/or on mechanical support or treatment with intravenous vasodilators, inotropes or vasopressors were excluded. Other exclusion criteria were concomitant or planned treatment with oral digoxin (could be randomized if the plasma concentration of digoxin at screening was <0.5 ng/ml); acute coronary syndrome or stroke within the past 3 months; coronary artery bypass graft or percutaneous coronary intervention within the past month or planned in the next month; life-threatening ventricular arrhythmia or implantable cardioverter defibrillator shock within the past month; sustained ventricular tachycardia in the last 3 months or uncontrolled arrhythmia; fever >38° C.; estimated glomerular filtration rate (eGFR)<30 ml/min/m²; serum potassium >5.3 or <3.5 mmol/L; stroke or transient ischaemic accident within the past 3 months; and acute respiratory distress syndrome.

The patients were randomized centrally, using an interactive response technology, to receive istaroxime or placebo at a ratio of 1:1. Study medication was supplied in uniquely-numbered kits containing identical vials of lyophilized powder (istaroxime plus lactose), reconstituted by adding 5 ml saline to the vial. Istaroxime was administered as a continuous infusion 1.0 μg/kg/min for 24 h. The infusion rate could be decreased at the discretion of the investigator based on the development of tolerability issues (such as nausea), significant bradycardia, or greater than desired BP elevation. The original protocol had a target and maximum dose infusion of 1.5 μg/kg/min; however, after 26 of the 60 patients were recruited, the sponsor and executive steering committee amended the protocol to limit the dose of istaroxime to 1.0 μg/kg/min, after which all patients were to receive a target and maximum dose of 1.0 μg/kg/min of istaroxime.

The primary efficacy endpoint was the area under the curve representing the change in SBP from baseline, start of study drug infusion, through 6 h (SBP AUC). The secondary endpoints included SBP AUC through 24 h; changes from baseline in SBP (particularly at 6 and 24 h), diastolic blood pressure (DBP) and mean arterial pressure (MAP); changes from baseline in heart rate (HR); treatment failure score (based on death, circulatory, respiratory, or renal mechanical support or intravenous inotrope or vasopressor treatment, and changes in SBP); treatment failure defined as death or need for circulatory, respiratory, or renal mechanical support or intravenous inotrope or vasopressor treatment; increase from baseline in SBP >5% and/or >10 mmHg; changes in quality of life measured by the EuroQol 5 Dimension 5 Level (EQ-5D-5L); change from baseline to 24 h in echocardiography parameters; changes in troponin and N-terminal pro-B-type natriuretic peptide (NT-proBNP); hospital readmission for heart failure and for any cause by day 30; in-hospital worsening heart failure to day 5; and length of hospital stay. In-hospital worsening heart failure was defined as worsening signs and/or symptoms of heart failure since the previous assessment that required an intensification of intravenous therapy for heart failure or mechanical ventilatory, renal, or circulatory support. The safety endpoints were assessed throughout the study and included the incidence of adverse events; changes in vital signs and in 12-lead electrocardiogram (ECG) parameters; incidence of clinically or haemodynamically significant episodes of supraventricular or ventricular arrhythmias detected by continuous ECG monitoring; standard laboratory parameters; renal function measures; cardiac troponin I or T; and mortality through day 30. The study is summarized in Table 1 and FIG. 1.

### Statistical Analysis

The primary efficacy analysis population was a modified intention-to-treat (mITT) population defined as subjects who received study treatment (any istaroxime or placebo infused to patient) and had at least one post-baseline BP assessment. Supportive efficacy analyses were conducted in an ITT population including all randomized patients, and a per-protocol population including subjects who received study drug infusion without an excluding protocol violation. Safety analyses included all patients who received any study medication. As all patients who were randomized received study treatment and had at least one post-baseline BP assessment, the mITT, ITT, and safety populations were the same. Because the dosing regimen was changed after the trial was underway, patients in the active group were classified by the maximum istaroxime dose received (all <1.0 vs. any >1.0 μg/kg/min), and additional pairwise comparisons were made.

Unadjusted results for continuous variables are presented as the mean and standard deviation while adjusted changes are presented as least square mean change and corresponding standard error (SE). Frequencies are presented for categorical variables. Sites that enrolled fewer than six patients were pooled and treated as one site for adjustment by pooled site. Frequencies are reported for categorical variables and binary clinical endpoints.

The primary endpoint, SBP AUC through hour 6, was computed by trapezoidal rule. Treatment groups were compared using ANCOVA with baseline value, pooled site, and treatment in the model. Changes in creatinine clearance, heart rate, MAP, natriuretic peptides, and troponin were compared between treatment groups using mixed model repeated measures with baseline value, treatment, pooled study site, time, and treatment by time interaction in the models. Changes in EuroQol visual analogue scale from baseline to 96 h and 30 days, and changes in echocardiographic measures at 24 and 30 h were compared between treatment groups at each time point using ANCOVA with baseline value, treatment, and pooled study site in the models. Van Elteren test stratified by pooled study centre was used to compare groups regarding treatment failure score (through 24 h), length of hospital stay, stay in intensive care or coronary care units (ICU/CCU), days alive out of the hospital, and days alive out of acute care through day 30. The proportion of subjects who sustained treatment failure, with any hospital readmission through day 30, or with increases from baseline in SBP >5% and >10 mmHg at a timepoint between 4 and 6 h after dosing, were compared using the Cochran-Mantel-Haenszel (CMH) test controlling for pooled sites. Two-sided p<0.05 was considered statistically significant. No adjustments for multiple testing were made. SAS version 9.4 (SAS Institute, Inc., Cary, NC, USA) was used for analyses.

### Results

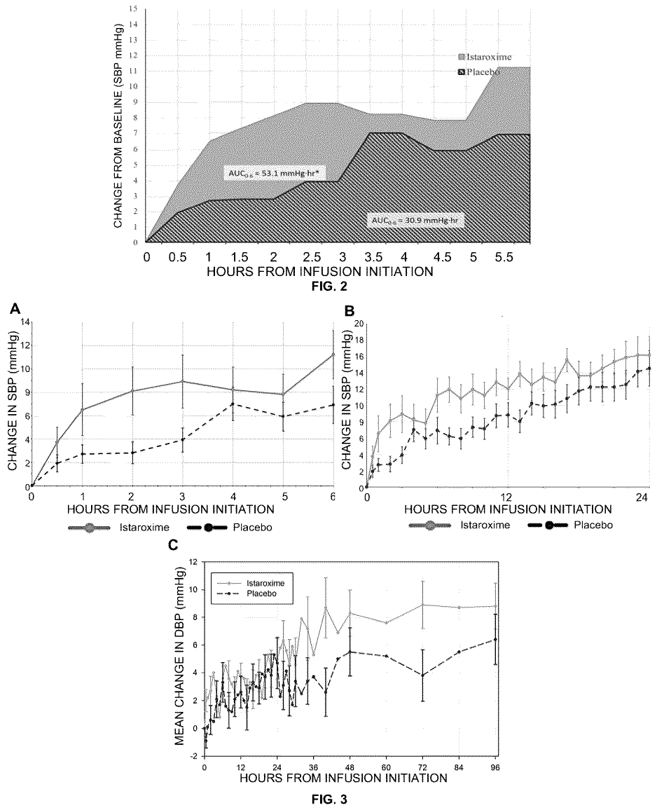

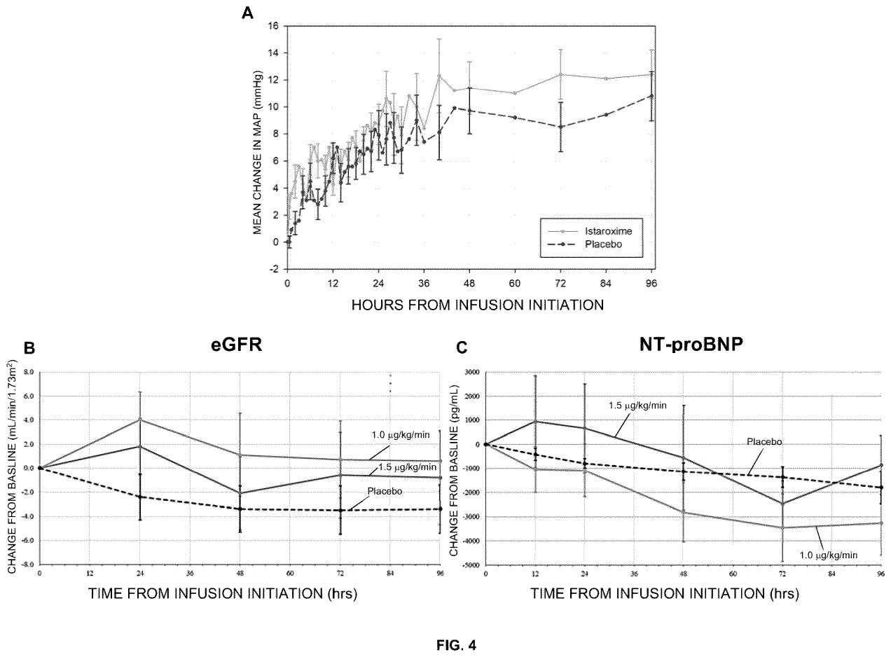

The primary endpoint was the AUC representing the change in SBP from time of infusion start to hour 6. FIGS. 2-4 summarize the results of this analysis. The adjusted mean 6 h AUC was 53.1 (SE 6.88) mmHg·h in the istaroxime-treated patients versus 30.9 (SE 6.76) mmHg·h in the placebo group (p=0.017), which was an increase of 72% (see FIG. 2). Moreover, the adjusted mean 24 h SBP AUC was 291.2 (SE 27.5) mmHg·h in the istaroxime group versus 208.7 (SE 27.0) mmHg·h in the placebo group (p=0.025), which as an increase of 40% (data not shown). As shown in FIG. 3A, the adjusted SBP increase at 6 h was 12.3 (SE 1.71) mmHg in the istaroxime-treated group versus 7.5 (SE 1.64) mmHg in the placebo group (p=0.045). The corresponding adjusted changes in SBP at 24 h were 17.1 (SE 2.36) mmHg and 15.1 (SE 2.25) mmHg in the istaroxime group versus placebo (p=0.543) as shown in FIG. 3B. Additionally, increases were noted in DBP (see FIG. 3C) and MAP (see FIG. 4A), the latter two persisting beyond the 24 h of study drug administration. Changes in laboratory values between the istaroxime and placebo-treated patients for eGFR and NT-proBNP are shown in FIGS. 4B and 4C, respectively.

Echocardiographic changes during the first 24 h of the study are presented in Table 3. Among the echocardiographic measures assessed, there were significant improvements at 24 h in some measures after adjustment that included cardiac index (+0.16+0.1 vs.-0.06+0.1 L/min/m²; p=0.016), left atrial area (−1.8+0.5 vs. 0.0+0.5 cm²; p=0.008), left ventricular end-systolic volume (−8.7+4.2 vs. 3.3+4.2 ml; p=0.034) and left ventricular end-diastolic volume (−6.5+4.9 vs. 5.6+4.8 ml; p=0.061) in the istaroxime-treated patients as compared to the placebo-treated patients.

TABLE 3
Echocardiographic changes during the first 24 h.

| | Istaroxime (n = 29) | | | | Placebo (n = 31) | | | | |
|---|---|---|---|---|---|---|---|---|---|
| | | | Change from | | | | Change from | | p- |
| Parameter/Time Point | Observed | | Baseline | | Observed | | Baseline | | value |
| Cardiac Index (L/min/m²) | | | | | | | | | |
| Baseline (pre-dose) | 1.9 | (0.57) | | | 2.0 | (0.63) | | | |
| 24 h Post-Treatment | 2.1 | (0.60) | 0.156 | (0.065) | 1.9 | (0.61) | −0.057 | (0.067) | 0.016 |
| Cardiac Output (L/min) | | | | | | | | | |
| Baseline (pre-dose) | 3.63 | (1.12) | | | 3.87 | (1.30) | | | |
| 24 h Post-Treatment | 3.93 | (1.19) | 0.23 | (0.13) | 3.93 | (1.35) | −0.02 | (0.13) | 0.141 |
| E/A Ratio | | | | | | | | | |
| Baseline (pre-dose) | 1.9 | (1.27) | | | 1.8 | (0.84) | | | |
| 24 h Post-Treatment | 1.5 | (1.06) | −0.380 | (0.172) | 1.8 | (0.93) | 0.049 | (0.187) | 0.090 |
| E/Ea Ratio | | | | | | | | | |
| Baseline (pre-dose) | 12.1 | (5.44) | | | 13.1 | (5.45) | | | |
| 24 h Post-Treatment | 12.2 | (6.09) | −0.615 | (0.893) | 11.2 | (5.42) | −2.139 | (0.929) | 0.195 |
| Ejection Time (ms) | | | | | | | | | |
| Baseline (pre-dose) | 223.5 | (62.12) | | | 231.8 | (51.66) | | | |
| 24 h Post-Treatment | 219.0 | (55.80) | −2.807 | (6.197) | 230.6 | (59.33) | 2.201 | (6.288) | 0.545 |
| Inferior Vena Cava | | | | | | | | | |
| Diameter (mm) | | | | | | | | | |
| Baseline (pre-dose) | 22.5 | (4.56) | | | 22.3 | (5.73) | | | |
| 24 h Post-Treatment | 19.5 | (5.49) | −2.503 | (0.700) | 20.0 | (6.06) | −2.080 | (0.695) | 0.647 |
| Left Atrial Area (cm²) | | | | | | | | | |
| Baseline (pre-dose) | 29.5 | (6.62) | | | 29.0 | (8.19) | | | |
| 24 h Post-Treatment | 27.1 | (6.27) | −1.817 | (0.501) | 29.0 | (7.12) | 0.004 | (0.500) | 0.008 |
| Left Atrial Volume (ml) | | | | | | | | | |
| Baseline (pre-dose) | 105.9 | (46.63) | | | 118.0 | (62.57) | | | |
| 24 h Post-Treatment | 95.2 | (39.70) | −7.440 | (1.876) | 112.5 | (61.40) | −3.170 | (1.906) | 0.095 |
| Left Ventricular End- | | | | | | | | | |
| Diastolic Volume (ml) | | | | | | | | | |
| Baseline (pre-dose) | 202.0 | (78.19) | | | 202.3 | (60.45) | | | |
| 24 h Post-Treatment | 194.3 | (79.36) | −6.501 | (4.864) | 206.3 | (65.94) | 5.641 | (4.755) | 0.061 |
| Left Ventricular End- | | | | | | | | | |
| Systolic Volume (ml) | | | | | | | | | |
| Baseline (pre-dose) | 146.6 | (65.59) | | | 151.2 | (54.98) | | | |
| 24 h Post-Treatment | 135.7 | (70.57) | −8.692 | (4.202) | 151.2 | (59.83) | 3.312 | (4.186) | 0.034 |
| Left Ventricular | | | | | | | | | |
| Ejection Fraction (%) | | | | | | | | | |
| Baseline (pre-dose) | 26.6 | (7.03) | | | 25.7 | (7.40) | | | |
| 24 h Post-Treatment | 29.5 | (7.64) | 2.678 | (0.739) | 27.6 | (8.61) | 1.383 | (0.723) | 0.184 |
| Left Ventricular End- | | | | | | | | | |
| Diastolic Diameter (mm) | | | | | | | | | |
| Baseline (pre-dose) | 62.5 | (8.25) | | | 65.7 | (9.12) | | | |
| 24 h Post-Treatment | 61.3 | (8.64) | −0.668 | (0.438) | 64.1 | (9.30) | −0.807 | (0.434) | 0.811 |
| Left Ventricular End- | | | | | | | | | |
| Systolic Diameter (mm) | | | | | | | | | |
| Baseline (pre-dose) | 54.4 | (9.66) | | | 54.9 | (12.92) | | | |
| 24 h Post-Treatment | 52.8 | (9.89) | −0.719 | (1.457) | 56.0 | (9.68) | 2.252 | (1.464) | 0.125 |
| Mitral Regurgitation | | | | | | | | | |
| Baseline (pre-dose) | | | | | | | | | |
| None | 0 | (0%) | | | 0 | (0%) | | | |
| Mild | 8 | (28%) | | | 15 | (48%) | | | |
| Moderate | 21 | (72%) | | | 16 | (52%) | | | |
| 24 h Post Treatment | | | | | | | | | |
| None | 1 | (4%) | | | 0 | (0%) | | | |
| Mild | 11 | (39%) | | | 14 | (47%) | | | |
| Moderate | 16 | (57%) | | | 16 | (53%) | | | |
| Pulmonary Artery | | | | | | | | | |
| Systolic Pressure (mmHg) | | | | | | | | | |
| Baseline (pre-dose) | 44.9 | (14.97) | | | 46.0 | (18.38) | | | |
| 24 h Post-Treatment | 36.8 | (14.58) | −7.084 | (1.878) | 39.4 | (16.12) | −4.790 | (1.798) | 0.348 |
| Stroke Volume Index (ml/m²) | | | | | | | | | |
| Baseline (pre-dose) | 24.4 | (6.41) | | | 23.7 | (6.54) | | | |
| 24 h Post-Treatment | 28.3 | (9.19) | 4.001 | (1.346) | 24.8 | (6.96) | 0.930 | (1.355) | 0.088 |
| Tricuspid Annular Plane | | | | | | | | | |
| Systolic Excursion (mm) | | | | | | | | | |
| Baseline (pre-dose) | 15.1 | (5.34) | | | 13.7 | (4.39) | | | |
| 24 h Post-Treatment | 15.9 | (5.02) | 0.0860 | (0.493) | 14.1 | (3.83) | 0.492 | (0.484) | 0.573 |

As shown above, istaroxime treatment led to an increase in SBP from start of treatment to 24 h by both AUC at 6 and 24 h. Of note, this study is the first to show a beneficial effect on BP in CS patients with any non-adrenergic drug, and hence without effects on pulse. Some improvements in echocardiographic measures were observed including increases in cardiac index and reduction in left ventricular and atrial dimensions. These findings suggest that istaroxime improved cardiac function in this patient population. The concomitant increase in both cardiac index and BP is unique and has not been observed with any previous intravenous drugs administered to patients with CS. This improvement can potentially allow for faster stabilization of patients with CS and earlier initiation of other lifesaving therapies.

1-17. (canceled)

18. A pharmaceutical composition for treatment of pre-cardiogenic shock or cardiogenic shock in an individual, the pharmaceutical composition comprising: (i) a pharmaceutically acceptable carrier; and (ii) istaroxime or a pharmaceutically acceptable salt, solvate, or hydrate thereof, wherein the istaroxime or pharmaceutically acceptable salt, solvate, or hydrate thereof is formulated for administration by intravenous infusion at a dose of about 0.2 μg/kg/min to about 2.0 g/kg/min for at least about 6 hours.

19. The pharmaceutical composition of claim 18, wherein the istaroxime or pharmaceutically acceptable salt, solvate, or hydrate thereof is formulated for administration by intravenous infusion at a dose of about 1.0 μg/kg/min to about 1.5 g/kg/min for at least about 24 hours.

20-23. (canceled)

24. The pharmaceutical composition of claim 18, further comprising saline as the pharmaceutically acceptable carrier and wherein the istaroxime is reconstituted from a lyophilized powder comprising the istaroxime admixed with a bulking agent.

25-27. (canceled)

28. A method of treating or preventing early cardiogenic shock or cardiogenic shock in an individual, the method comprising:
a) providing an individual with acute heart failure;
b) determining that the individual has a systolic blood pressure of less than about 90 mmHg for a first period of time of at least about 1 hour; and
c) administering to the individual a therapeutically effective amount of a pharmaceutical composition comprising: (i) a pharmaceutically acceptable carrier; and (ii) istaroxime or a pharmaceutically acceptable salt, solvate, or hydrate thereof, wherein the istaroxime or pharmaceutically acceptable salt, solvate, or hydrate thereof is administered at a dose of about 0.2 μg/kg/min to about 2.0 μg/kg/min, and wherein the administering comprises intravenous infusion for a second period of time of at least about 3 hours; and
wherein the administering of the pharmaceutical composition results in an increase over baseline for both systolic heart function and cardiac output, the increase measurable by a third period of time following the start of the intravenous infusion, thereby treating or preventing early cardiogenic shock or cardiogenic shock in the individual.

29. The method of claim 28, further comprising measuring one or more parameters of heart function in the individual, wherein the one or more parameters of heart function comprises blood pressure.

30. The method of claim 29, wherein the blood pressure is systolic blood pressure.

31. The method of claim 30, wherein the administering of the pharmaceutical composition results in an increase over baseline for the systolic blood pressure, and wherein the third period of time is about 6 hours.

32. The method of claim 30, wherein the blood pressure is measured as systolic blood pressure area under the curve and administering of the pharmaceutical composition results in an increase over baseline for the systolic blood pressure area under the curve, and wherein the third period of time is about 6 hours.

33. The method of claim 29, wherein the one or more parameters of heart function in the individual additionally includes cardiac index, left atrial area, stroke volume, left ventricular end-systolic volume, left ventricular end-diastolic volume, or any combination thereof.

34. The method of claim 28, wherein the acute heart failure is acute decompensated heart failure without evidence of end-organ hypoperfusion or acute coronary syndrome.

35. The method of claim 28, further comprising determining that the individual has a heart rate of about 75 beats/minute (bpm) to about 150 bpm or about 60 bpm to about 150 bpm if the individual has been administered a beta blocker.

36. The method of claim 28, wherein the istaroxime or pharmaceutically acceptable salt, solvate, or hydrate thereof is administered at a dose of about 1.0 μg/kg/min to about 1.5 μg/kg/min.

37. The method of claim 28, wherein the first period of time is at least about 2 hours.

38. The method of claim 28, wherein the second period of time is selected from at least about 6 hours and at least about 24 hours.

39. (canceled)

40. The method of claim 28, wherein the individual is a human aged at least 18 years old.

41. The method of claim 28, wherein the individual has at least one of dyspnea at rest or minimal exertion, congestion on chest x-ray or lung ultrasound with brain natriuretic peptide of less than about 400 μg/mL or N-terminal pro b-type Natriuretic Peptide of at least 1,400 μg/ml, or left ventricular ejection fraction of less than about 40%, or echocardiogram confirming ejection fraction of less than about 40%.

42. The method of claim 29, wherein the one or more parameters of heart function further comprise heart rate (HR), diastolic relaxation, systolic contraction, dyspnea, chest congestion, diastolic blood pressure (DBP), systolic blood pressure (SBP), creatine clearance, deceleration slope, mitral inflow velocity, mean arterial pressure, brain natriuretic peptide levels (BNP), NT-pro-BNP levels, troponin levels, venous lactate levels, echocardiographic measurements, left ventricle end diastolic diameter (EDD), left ventricle end systolic diameter (ESD), left ventricle end diastolic volume (EDV), left ventricle end systolic volume (ESV), left atrium diameter (LAD), left atrium area (LAA), left atrium volume (LAV), E wave, A wave, E wave deceleration time (EDT), E/A ratio, Ea, Aa, E/Ea ratio, left ventricle ejection fraction (LVEF), Sa, stroke volume (SV), cardiac output (CO), stroke volume index (SVI), cardiac index (CI), pulmonary arterial systolic pressure (PASP), tricuspid annular plane systolic excursion (TAPSE), right ventricle Sa, mitral regurgitation (MR), inferior vena cava diameter (IVC), or any combination thereof.

43. The method of claim 28, wherein the individual is undergoing a therapeutic treatment for pre-cardiogenic shock or cardiogenic shock with one or more other further-therapeutically active ingredients.

44. The method of claim 43, wherein the other further therapeutically active ingredients are selected from the group consisting of ACE inhibitors, ARBs, diuretics, Ca channel blockers, beta-blockers, digitalis, NO donors, vasodilators, SERCA2a stimulators, Angiotensin receptor/neprilysin inhibitors (ARNIs), myosin filament activators, recombinant relaxin-2 mediators, recombinant NP protein, activators of the soluble Guanylate Cyclase (sGC), and beta-arrestin ligand of Angiotensin II receptor.

45. The method of claim 44, wherein:
(a) the diuretic, is selected from the group consisting of furosemide, bumetanide, torasemide, metolazone, an aldosterone antagonist, and a thiazide; or
(b) the ACE inhibitor is Lisinopril or Ramipril; or
(c) the Angiotensin II receptor blocker is selected from the group consisting of valsartan, candesartan, olmesartan, telmisartan, and losartan; or
(d) the ARNI is sacubitril; or
(e) the beta-blocker is carvedilol or metoprolol; or
(f) the vasodilator is selected from the group consisting of hydralazine, amlodipine, felodipine, diltiazem, and verapamil.

46. (canceled)

47. A method of treating pre-cardiogenic shock in an individual, the method comprising:
a) providing an individual with pre-cardiogenic shock with acute heart failure, wherein the pre-cardiogenic shock comprises a systolic blood pressure of less than about 90 mm Hg without end-organ hypoperfusion or myocardial infarction;
b) administering to the individual a therapeutically effective amount of a pharmaceutical composition comprising: (i) a pharmaceutically acceptable carrier; and (ii) istaroxime or a pharmaceutically acceptable salt, solvate, or hydrate thereof, wherein the istaroxime or pharmaceutically acceptable salt, solvate, or hydrate thereof is administered at a dose of about 0.2 μg/kg/min to about 2.0 μg/kg/min, and wherein the administering comprises intravenous infusion for at least about 6 hours; and
c) measuring one or more parameters of heart function in the individual, wherein the one or more parameters of heart function comprises blood pressure;
wherein the administering of the pharmaceutical composition results in an increase over baseline for the blood pressure, the increase measurable by 6 hours following the start of the intravenous infusion, thereby treating pre-cardiogenic shock in the individual.

48. The method of claim 47, wherein the blood pressure is systolic blood pressure, and the administration of the pharmaceutical composition results in an increase over baseline for the systolic blood pressure.

49. The method of claim 47, wherein the blood pressure is measured as systolic blood pressure area under the curve, and the administration of the pharmaceutical composition results in an increase over baseline for the systolic blood pressure area under the curve.

50. The method of claim 47, wherein the one or more parameters of heart function in the individual additionally includes cardiac index, left atrial area, stroke volume, left ventricular end-systolic volume, left ventricular end-diastolic volume, or any combination thereof, wherein the administering of the pharmaceutical composition results in:
(a) increased cardiac index; or
(b) decreased left atrial area; or
(c) increased stroke volume; or
(d) decreased left ventricular end-diastolic volume; or
(e) decreased left ventricular end-diastolic volume; or
(f) any combination of (a)-(e).

51. (canceled)

52. The method of claim 47, wherein the acute heart failure is acute decompensated heart failure.

53. (canceled)

54. The method of claim 47, wherein the istaroxime or pharmaceutically acceptable salt, solvate, or hydrate thereof is administered at a dose of about 1.0 g/kg/min to about 1.5 μg/kg/min.

55. The method of claim 47, wherein the administering comprises intravenous infusion for at least about 24 hours.

56. The method of claim 47, wherein the one or more parameters of heart function further comprise heart rate (HR), diastolic relaxation, systolic contraction, dyspnea, chest congestion, diastolic blood pressure (DBP), systolic blood pressure (SBP), creatine clearance, deceleration slope, mitral inflow velocity, mean arterial pressure, brain natriuretic peptide levels (BNP), NT-pro-BNP levels, troponin levels, venous lactate levels, echocardiographic measurements, left ventricle end diastolic diameter (EDD), left ventricle end systolic diameter (ESD), left ventricle end diastolic volume (EDV), left ventricle end systolic volume (ESV), left atrium diameter (LAD), left atrium area (LAA), left atrium volume (LAV), E wave, A wave, E wave deceleration time (EDT), E/A ratio, Ea, Aa, E/Ea ratio, left ventricle ejection fraction (LVEF), Sa, stroke volume (SV), cardiac output (CO), stroke volume index (SVI), cardiac index (CI), pulmonary arterial systolic pressure (PASP), tricuspid annular plane systolic excursion (TAPSE), right ventricle Sa, mitral regurgitation (MR), inferior vena cava diameter (IVC), or any combination thereof.

57-59. (canceled)
